(19) United States
(12) Patent Application Publication
(10) Pub. No.: US 2017/0242010 A1
(43) Pub. Date: Aug. 24, 2017

(54) Combo-Hepatitis Antigen Assays and Kits for Detection of Active Hepatitis Virus Infections

(71) Applicant: The Regents of the University of California, Oakland, CA (US)

(72) Inventor: Ke-Qin Hu, Irvine, CA (US)

(73) Assignee: The Regents of the University of California, Oakland, CA (US)

(21) Appl. No.: 15/114,262

(22) PCT Filed: Feb. 10, 2015
(86) PCT No.: PCT/US15/15092

§ 371 Date: Jul. 26, 2016

Related U.S. Application Data
(60) Provisional application No. 61/938,590, filed on Feb. 11, 2014.

(51) Int. Cl. G01N 33/576; C12Q 1/70; G01N 33/543

(52) U.S. Cl. CPC G01N 33/5767; G01N 33/5761; G01N 33/54386; C12Q 1/707; G01N 2333/02; G01N 2800/26; C12Q 2600/158

(57) ABSTRACT

Disclosed herein are assays, systems, and kits for the detection and diagnosis of hepatitis virus infections in subjects.

### BACKGROUND OF THE INVENTION

1. Field of the Invention

The present invention is directed to assays and assay systems and kits for detecting and diagnosing active hepatitis virus infections.

2. Description of the Related Art

Hepatitis C virus (HCV) infection affects approximately 170 million people worldwide, and 4-5 million people in the United States. HCV infection has been associated with chronic hepatitis C (CHC), cirrhosis, and hepatocellular carcinoma (HCC). Recent studies indicated HCV infection remains under screened and diagnosed that may result in delayed access to medical follow up and effective treatment in many subjects. In addition, the Centers for Disease Control and Prevention (CDC) have recommended that all individuals who were born in 1945-1965 be screened for HCV infection.

Since discovery of the HCV genome, a variety of anti-HCV antibody tests have been developed to screen for HCV infection. Anti-HCV antibody tests detect the presence of anti-HCV antibodies produced by HCV-infected individuals. Thus, anti-HCV antibody tests require time for an immune response and antibody formation. As such, anti-HCV antibody tests cannot be used for detecting acute HCV infections. Although the third generation anti-HCV antibody test has significantly improved sensitivity and specificity compared to prior generations of anti-HCV antibody tests, it cannot distinguish an ongoing active HCV infection from a prior HCV infection because of the anti-HCV antibodies that remain after clearance of the virus. Additionally, anti-HCV antibody tests have a high incidence of false negative results for subjects who are immunocompromised, receiving immunosuppressive therapy, or undergoing hemodialysis. Consequently, an additional test, e.g., a PCR assay for HCV RNA, is needed to confirm active HCV infection in those testing positive for HCV infection using an anti-HCV antibody test. Unfortunately, HCV RNA PCR tests are time consuming, expensive, and currently not recommended as a screening test for HCV infection.

Other HCV tests detect HCV Core Antigen (HCVcAg). HCVcAg exists in both complete HCV virions and HCV RNA-free core protein structure. HCVcAg is considered a marker of active HCV replication and is detectable earlier than anti-HCV antibodies. The major HCVcAg test systems are the Ortho HCV Core Ag EIA test (Ortho Clinical Diagnostics, Raritan, N.J.), the Architect HCVcAg test (Abbott Laboratories, Abbott Park, Ill.), and the Monolisa HCV Ag/Ab ULTRA assay (Bio-Rad, Hercules, Calif.).

The Ortho HCV Core Ag EIA test was originally developed as a blood donor screening test in combination with anti-HCV test to cover possible negative anti-HCV test results derived from the “window period” of seroconversion from anti-HCV negative to positive. It is an enzyme-linked immunosorbent assay (ELISA, or EIA) for the detection of hepatitis C core antigen in human serum or plasma. The assay utilizes several monoclonal antibodies specific to different regions of the HCV core antigen to coat microplate solid phase and capture HCVcAg present in the tested serum samples. After that, additional HCVcAg-specific monoclonal antibodies conjugated to horseradish peroxidase will then be used to detect the captured HCVcAg. The sensitivity of this test (HCM V2.0 assay) was reported to be 1.48 pg/mL of HCVcAg, corresponding to 9,707 IU/mL of HCV RNA (1). A literature review concluded a high rate of false negative results by this test in HCV RNA PCR-positive cases. Thus, the low sensitivity of Ortho HCV Core Ag EIA limits its clinical value.

The Architect HCVcAg test is a two-step chemiluminescent microparticle immunoassay (CMIA) for quantitative determination of HCVcAg in human serum and plasma samples. The assay uses acridinium labeled murine anti-HCV antibodies in the liquid phase and monoclonal anti-HCV coated paramagnetic microparticles as the solid phase. The reported detection limit of this test is at 3 fmol/L, or 0.06 pg/mL of HCVcAg with a dynamic HCVcAg quantification at range of 3.0-20,000 fmol/L. Clinically, this corresponds to serum HCV RNA levels in the range of 428-2700 IU/mL, depending on HCV genotyping. Although some studies reported that this test is highly sensitive, other studies also reported that its overall correlation to HCV RNA PCR results was only 79.7%, that could be as low as 19.7% in subjects with serum HCV RNA<3 log. It was also reported that in 9/405 (2.2%) subjects with undetectable HCV RNA, HCVcAg was reported positive (HCVcAg>3 fmol/L), indicating false positive HCVcAg test results in these subjects. In addition, when serum HCV RNA is in very lower level (<15 IU/mL), more false positive results may occur. Furthermore, this test has to be automated via a special and expensive equipment supplied by the vendor, which is not easily adopted by routine laboratories for a broad clinical application, especially in developing countries. Currently, Architect HCV core antigen test is not approved in US and many other countries. Consequently, this HCVcAg test system is neither economic, sensitive and specific, nor practical, and will be difficult for wide clinical application.

The Monolisa HCV Ag-Ab ULTRA assay is a two-step ELISA for simultaneously detection of both anti-HCV and HCVcAg in human serum and plasma samples. It is based on a combination of an indirect test for anti-HCV and a sandwich test for HCVcAg detection. Although the Monolisa HCVAg-Ab ultra assay is reported with improved performance by simultaneously detecting both HCVcAg and anti-HCV antibodies, it cannot distinguish the HCV-Ab signal from HCVcAg signal, and therefore, it cannot differentiate ongoing active HCV infection from recovered or past HCV infection. Additionally, this test was designed to increase diagnostic rate for acute HCV infection, studies indicated approximately 29% subjects with acute HCV infection will be missed by this test due to relatively low sensitivity of HCVcAg component in this assay.

It should be noted, besides the low test sensitivity in samples with low HCV RNA load, one of the other main limitations of the current HCVcAg assays is a high rate of positivity in subjects positive for serum anti-HCV, but negative for serum HCV RNA by PCR tests. As these individuals typically have past, but not active HCV infection, a positive test for HCVcAg in these subjects should be considered false positivity. This results in the inability of the current HCVcAg tests to differentiate an active HCV infection from a past infection. In other words, when using the current HCVcAg tests, one cannot tell if a subject tested positive for HCVcAg is because of an active HCV infection or a past HCV infection.

Therefore, a need exists for a safe and convenient test that can be used to detect active hepatitis virus infection in a subject with sufficient sensitivity and specificity.

### SUMMARY OF THE INVENTION

In some embodiments, the present invention provides an assay for identifying a sample as containing one or more hepatitis virus antigens, which comprises contacting the sample with a plurality of antibodies that specifically bind the one or more hepatitis virus antigens, detecting the presence or absence of any hepatitis virus antigens bound to the antibodies of the plurality of antibodies, optionally measuring any hepatitis virus antigens bound to the antibodies of the plurality of antibodies, and identifying the sample as containing hepatitis virus antigens where hepatitis virus antigens bound to the antibodies of the plurality of antibodies are detected as being present, and identifying the sample as not containing hepatitis virus antigens where hepatitis virus antigens bound to the antibodies of the plurality of antibodies are absent. In some embodiments, the one or more hepatitis virus antigens are HCV antigens and/or hepatitis B Virus (HBV) antigens. In some embodiments, the hepatitis virus antigens comprise or consist of HBV surface antigen (HBsAg). In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a. In some embodiments, the sample is subjected to a condition that disassociates immune complexes prior to the detecting step. In some embodiments, the sample is not subjected to a condition that disassociates immune complexes prior to the detecting step. In some embodiments, the sample is urine. In some embodiments, the sample is whole blood, serum, or plasma. In some embodiments, the plurality of antibodies comprises a first antibody and a second antibody, said first and second antibodies specifically bind the same hepatitis virus antigen. In some embodiments, the assay further comprises mixing the sample with the plurality of antibodies to form a mixture and then contacting the mixture with a substrate having a capture reagent that specifically binds the plurality of antibodies, which may or may not be bound to the hepatitis virus antigens before the detecting step. In some embodiments, the detecting step comprises attaching a detectable label to each antibody of the plurality of antibodies.

In some embodiments, the present invention provides a method of diagnosing a subject as having an active hepatitis virus infection, which comprises diagnosing the subject as having an active hepatitis virus infection where a urine sample from the subject or another sample (e.g., a whole blood, serum, plasma sample, and the like) from the subject that has not been subjected to denaturing conditions has been identified as containing free hepatitis virus antigens using an assay of the present invention as described herein. In some embodiments, the assay is for identifying a sample as containing free hepatitis virus antigens, which comprises contacting the sample with a plurality of antibodies wherein each antibody in the plurality specifically binds a hepatitis virus antigen of a plurality of hepatitis virus antigens, detecting the presence or absence of any hepatitis virus antigens bound to the antibodies of the plurality of antibodies, optionally measuring any hepatitis virus antigens bound to the antibodies of the plurality of antibodies, and identifying the sample as containing free hepatitis virus antigens where hepatitis virus antigens bound to the antibodies of the plurality of antibodies are detected as being present, and identifying the sample as not containing free hepatitis virus antigens where hepatitis virus antigens bound to the antibodies of the plurality of antibodies are absent. In some embodiments, the hepatitis virus infection is hepatitis C virus (HCV) infection. In some embodiments, the hepatitis virus infection is hepatitis B virus (HBV) infection. In some embodiments, the one or more hepatitis virus antigens are HCV antigens and/or HBV antigens. In some embodiments, the hepatitis virus antigens comprise or consist of HBsAg. In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a. In some embodiments, the plurality of antibodies comprises a first antibody and a second antibody, said first and second antibodies specifically bind the same hepatitis virus antigen. In some embodiments, the assay further comprises mixing the sample with the plurality of antibodies to form a mixture and then contacting the mixture with a substrate having a capture reagent that specifically binds the plurality of antibodies, which may or may not be bound to the hepatitis virus antigens before the detecting step. In some embodiments, the detecting step comprises attaching a detectable label to each antibody of the plurality of antibodies. In some embodiments, the method is used to identify a subject, from a plurality of subjects, as having or not having an active hepatitis virus infection.

In some embodiments, the present invention is directed to a method for diagnosing a subject as having an active hepatitis virus infection or having had a past and cleared hepatitis virus infection, which comprises obtaining a first and a second sample from the subject, wherein at least the second sample is capable of having immune complexes, contacting the first sample, which is either not capable of having immune complexes and/or has not been subjected to conditions that disassociate immune complexes, with a plurality of antibodies wherein each antibody in the plurality specifically binds a hepatitis virus antigen of a plurality of hepatitis virus antigens, detecting the presence or absence of any hepatitis virus antigens bound to the antibodies of the plurality of antibodies, and optionally measuring any hepatitis virus antigens bound to the antibodies of the plurality of antibodies, contacting the second sample, which has been subjected to conditions that disassociate immune complexes, with the plurality of antibodies, detecting the presence or absence of any hepatitis virus antigens bound to the antibodies of the plurality of antibodies, and optionally measuring any hepatitis virus antigens bound to the antibodies of the plurality of antibodies, and diagnosing the subject as having an active hepatitis virus infection where hepatitis virus antigens bound to the antibodies of the plurality of antibodies are detected as being present in the first sample, and diagnosing the subject as having a past and cleared hepatitis virus infection where hepatitis virus antigens bound to the antibodies of the plurality of antibodies are detected in the second sample and no hepatitis virus antigens are detected in the first sample. For example, in some embodiments, the present invention is directed to a method for diagnosing a subject as having an active hepatitis virus infection or having had a past and cleared hepatitis virus infection, which comprises detecting and/or measuring free hepatitis virus antigens and detecting and/or measuring total hepatitis virus antigens in one or more samples form the subject and diagnosing the subject as having an active hepatitis virus infection where free hepatitis virus antigens are detected as being present, and diagnosing the subject as having a past and cleared hepatitis virus infection where total hepatitis virus antigens are detected as being present and free hepatitis virus antigens are absent. In some embodiments, the hepatitis virus infection is hepatitis C virus (HCV) infection. In some embodiments, the hepatitis virus infection is hepatitis B virus (HBV) infection. In some embodiments, the one or more hepatitis virus antigens are HCV antigens and/or HBV antigens. In some embodiments, the hepatitis virus antigens comprise or consist of HBsAg. In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a. In some embodiments, the first and second samples are whole blood, serum, or plasma. In some embodiments, the first sample is urine and the second sample is whole blood, serum, or plasma. In some embodiments, the first and second samples may be aliquots of the same specimen. In some embodiments, the plurality of antibodies comprises a first antibody and a second antibody, said first and second antibodies specifically bind the same hepatitis virus antigen. In some embodiments, the assay further comprises mixing the sample with the plurality of antibodies to form a mixture and then contacting the mixture with a substrate having a capture reagent that specifically binds the plurality of antibodies, which may or may not be bound to the hepatitis virus antigens before the detecting step. In some embodiments, the detecting step comprises attaching a detectable label to each antibody of the plurality of antibodies.

In some embodiments, the present invention is directed to a method of monitoring a subject who had, has, or may have an active hepatitis virus infection, which comprises obtaining a first and a second sample from the subject, wherein at least the second sample is capable of having immune complexes, at a first point in time, contacting the first sample, which is either not capable of having immune complexes and/or has not been subjected to conditions that disassociate immune complexes, with a plurality of antibodies wherein each antibody in the plurality specifically binds a hepatitis virus antigen of a plurality of hepatitis virus antigens, detecting the presence or absence of any hepatitis virus hepatitis virus bound to the antibodies of the plurality of antibodies, and optionally measuring any hepatitis virus antigens bound to the antibodies of the plurality of antibodies, contacting the second sample, which has been subjected to conditions that disassociate immune complexes, with the plurality of antibodies, detecting the presence or absence of any hepatitis virus antigens bound to the antibodies of the plurality of antibodies, and optionally measuring any hepatitis virus antigens bound to the antibodies of the plurality of antibodies, obtaining a third and a fourth sample from the subject, wherein at least the fourth sample is capable of having immune complexes, at a second point in time, contacting the third sample, which is either not capable of having immune complexes and/or has not been subjected to conditions that disassociate immune complexes, with a plurality of antibodies wherein each antibody in the plurality specifically binds an hepatitis virus antigen of a plurality of hepatitis virus antigens, detecting the presence or absence of any hepatitis virus antigens bound to the antibodies of the plurality of antibodies, and optionally measuring any hepatitis virus antigens bound to the antibodies of the plurality of antibodies, contacting the fourth sample, which has been subjected to conditions that disassociate immune complexes, with the plurality of antibodies, detecting the presence or absence of any hepatitis virus antigens bound to the antibodies of the plurality of antibodies, and optionally measuring any hepatitis virus antigens bound to the antibodies of the plurality of antibodies, and calculating the differences in hepatitis virus antigens bound to the antibodies of the plurality of antibodies between the first, second, third, and fourth samples. For example, in some embodiments, the presence or absence of and/or amounts of free hepatitis virus antigens and total hepatitis virus antigens in one or more samples from a subject at a first time period are compared with the presence or absence of and/or amounts of free hepatitis virus antigens and total hepatitis virus antigens in one or more samples from the subject at a second time period. In some embodiments, the ratio of free and total hepatitis antigens from the first time period is compared with the ratio of the free and total hepatitis antigens from the second time period. In some embodiments, the hepatitis virus infection is hepatitis C virus (HCV) infection. In some embodiments, the hepatitis virus infection is hepatitis B virus (HBV) infection. In some embodiments, the one or more hepatitis virus antigens are HCV antigens and/or HBV antigens. In some embodiments, the hepatitis virus antigens comprise or consist of HBsAg. In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a. In some embodiments, the samples are whole blood, serum, or plasma. In some embodiments, the first and third samples are urine and the second and fourth samples are whole blood, serum, or plasma. In some embodiments, the first and second samples are aliquots of the same specimen. In some embodiments, the third and fourth samples are aliquots of the same specimen. In some embodiments, the plurality of antibodies comprises a first antibody and a second antibody, said first and second antibodies specifically bind the same hepatitis virus antigen. In some embodiments, the assay further comprises mixing the sample with the plurality of antibodies to form a mixture and then contacting the mixture with a substrate having a capture reagent that specifically binds the plurality of antibodies, which may or may not be bound to the hepatitis virus antigens before the detecting step. In some embodiments, the detecting step comprises attaching a detectable label to each antibody of the plurality of antibodies.

In some embodiments, the present invention provides an immunoassay for an analyte in a test sample, which comprises mixing the test sample with one or more detection antibodies, which specifically bind the analyte, and then contacting the mixture with an assay substrate having capture antibodies, which specifically bind the analyte coated or immobilized thereon the surface of the assay substrate. In some embodiments, the immunoassay is an enzymatic immunoassay.

In some embodiments, the present invention provides a lateral flow test assay for an analyte in a test sample, which comprises mixing the test sample with detection antibodies, which specifically bind the analyte, conjugated with a detectable label, e.g., colloid gold, and then loading the mixture on a test strip having capture antibodies, which specifically bind the analyte, immobilized on a test line and antibodies, which specifically bind the detection antibodies, immobilized on a control line that is downstream of the test line.

Subjects according to the present invention are mammalian subjects, e.g., human subjects. In some embodiments, the subjects are in need of an assay according to the present invention. A subject in need of an assay according to the present invention include those who are suspected of having an active and/or past hepatitis virus infection and those who have been exposed to a hepatitis virus. In some embodiments, the one or more hepatitis virus antigens are HCV antigens and/or HBV antigens. In some embodiments, the hepatitis virus antigens comprise or consist of HBsAg. In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a.

In some embodiments, the present invention provides a lateral flow test substrate having a sample loading area (e.g., where the sample is first contacted with the lateral flow test substrate), a test area, and a control area, wherein a capture reagent is immobilized in the test area, said capture reagent is a plurality of antibodies wherein each antibody in the plurality specifically binds a hepatitis virus antigen of a plurality of hepatitis virus antigens. In some embodiments, the one or more hepatitis virus antigens are HCV antigens and/or HBV antigens. In some embodiments, the hepatitis virus antigens comprise or consist of HBsAg. In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a.

In some embodiments, the present invention provides a kit comprising a container containing a plurality of antibodies wherein each antibody in the plurality specifically binds a hepatitis virus antigen of a plurality of hepatitis virus antigens, and a substrate having coated or immobilized thereon a capture reagent that specifically binds the plurality of antibodies, the plurality of hepatitis virus antigens, or both. In some embodiments, the one or more hepatitis virus antigens are HCV antigens and/or HBV antigens. In some embodiments, the hepatitis virus antigens comprise or consist of HBsAg. In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a. In some embodiments, the plurality of antibodies is a composition having a concentration and/or purity of the plurality of antibodies that is not found naturally. In some embodiments, the kits further comprise a monoclonal or polyclonal antibody having a detectable label or conjugate attached thereto, said monoclonal or polyclonal antibody specifically binds the plurality of antibodies.

In some embodiments, the present invention provides a kit comprising a lateral flow test substrate having a sample loading area (e.g., where the sample is first contacted with the lateral flow test substrate), a test area, and a control area, wherein a capture reagent is immobilized in the test area, said capture reagent is a plurality of antibodies wherein each antibody in the plurality specifically binds a hepatitis virus antigen of a plurality of hepatitis virus antigens packaged together with a detection reagent. In some embodiments, the one or more hepatitis virus antigens are HCV antigens and/or HBV antigens. In some embodiments, the hepatitis virus antigens comprise or consist of HBsAg. In some embodiments, the hepatitis virus antigens comprise or consist of HCV antigens. In some embodiments, the HCV antigens are selected from the group consisting of HCVcAg, E1, E2, NS2, NS3, NS4a, NS4b, NS5a, and NS5b. In some embodiments, the HCV antigens are selected from the group consisting of HCVcAg, E1, E2, NS2, NS3, NS4a, NS4b, NS5a, and NS5b, and at least one of the HCV antigens is HCVcAg. In some embodiments, the HCV antigens comprise, consist essentially of, or consist of HCVcAg, NS3, NS4b, and NS5a. In some embodiments, the plurality of antibodies is a composition having a concentration and/or purity of the plurality of antibodies that is not found naturally. In some embodiments, the kits further comprise a monoclonal or polyclonal antibody having a detectable label attached thereto, said monoclonal or polyclonal antibody specifically binds the plurality of antibodies.

In embodiments where the hepatitis virus antigens consist essentially of a plurality of antigens, e.g., HCVcAg, NS3, NS4b, and NS5a, the phrase “consist essentially of” means that the assays, systems, and kits may include the detection of other antigens, which may or may not be hepatitis virus antigens, so long as the detection of the other antigens does not adversely impact the detection of antigens in the plurality of antigens, e.g., HCVcAg, NS3, NS4b, and NS5a.

In embodiments of the present invention, the detection of one or more hepatitis virus antigens that are bound to one or more antibodies may be by directly detecting the bound hepatitis virus antigen itself or indirectly by detecting the one or more antibodies bound to the hepatitis virus antigen. For example, a labeled antibody that specifically binds a given hepatitis virus antigen may be used to directly detect the given hepatitis virus antigen. Alternatively, a labeled antibody that specifically binds the antibodies bound to the antigen may be used for indirect detection.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1 schematically shows how a HCV polyprotein precursor is translated from HCV genome and then cleaved to different HCV structural and nonstructural proteins. C: core protein; E: envelop protein; NS: nonstructural proteins.

FIG. 2 is a schematic diagram of an immunochromatographic strip used for the LFT assays exemplified herein. A. Sample Pad; B. Backing card; C. Conjugate pad; D. Capture antibody conjugated with colloid gold particles; E. Nitrocellulose membrane; F. Test line; G. Control line; and H. Absorbent pad.

FIG. 3A is a bar graph showing that, in serum samples, the combo-HCV-Ags EIA assay (combo) has significantly increasing OD values, and hence, sensitivity, as compared with EIA assays detecting HCVcAg alone (core). These also confirmed the presence of HCV NS3, NS4b, and NS5a antigens besides HCVcAg in the blood samples during HCV infection. Negative: negative control using a serum specimen with negative anti-HCV and serum HCV RNA by PCR assay; S: test serum specimens with positive serum HCV RNA by PCR assay. The first bars in each set are “core”, the second bars in each set are “combo”.

FIG. 3B is a bar graph showing that, in urine samples, the combo-HCV-Ags EIA assay (combo) has significantly increasing OD values, and hence, sensitivity, as compared with EIA assays detecting HCVcAg alone (core). These also confirmed the presence of HCV NS3, NS4b, and NS5a antigens besides HCVcAg in the urine samples during HCV infection. Negative: negative control using a urine specimen with negative anti-HCV and serum HCV RNA by PCR assay; S: test urine specimens with positive serum HCV RNA by PCR assay. The first bars in each set are “core”, the second bars in each set are “combo”.

FIG. 4 is a bar graph showing that the addition of an additional HCVcAg mAb improved the sensitivity of the combo-HCV-Ags EIA assay (Example 2) when testing serum specimens. “1 Core”=EIA assay for HCVcAg alone using one HCVcAg mAb; “1 Core+Combo”=combo-HCV-Ags EIA assay employing one HCVcAg mAb+antibodies against NS3, NS4b, and NS5a; “2 Core”=EIA assay for HCVcAg alone using a first HCVcAg mAb and a second HCVcAg mAb; and “2 Core+Combo”=combo-HCV-Ags EIA assay employing a first HCVcAg mAb and a second HCVcAg mAb+antibodies against NS3, NS4b, and NS5a. PBS: using PBS buffer; Negative control: using a serum specimen with negative anti-HCV and serum HCV RNA by PCR assay; Sample: test serum specimens with positive serum HCV RNA by PCR assay.

FIG. 5, Panels A-D, shows besides HCVcAg, pre-treatment of HCV RNA-positive serum specimens with denaturation also increases the measured OD values or sensitivity by the EIA for HCVcAg (Panel A), NS3 (Panel B), NS4b (Panel C), and NS5a (Panel D). These also confirmed the presence of the immune complexes containing each of all these HCV-Ags in the blood specimens during HCV infection. Treated sample: the test serum samples (S1-S3) were pre-treated with denaturation (Example 2, with step 5); Non-treated sample: the test serum samples (S1-S3) were not pre-treated (Example 2, omitting step 5).

FIG. 6A is a bar graph showing the detection limit (equivalent to serum HCV RNA of about 188 IU/mL by PCR) of the combo-HCV-Ags EIA assay in a serum sample testing serial dilutions.

FIG. 6B is a bar graph showing the detection limit (equivalent to serum HCV RNA of about 328 IU/mL by PCR) of the combo-HCV-Ags EIA assay in another serum sample testing serial dilutions.

FIG. 6C is a bar graph showing the detection limit (equivalent to serum HCV RNA of about 250 IU/mL by PCR) of the combo-HCV-Ags EIA assay for different HCV genotypes. The graph represents an average of 5 samples from subjects infected by different HCV genotypes.

FIG. 7A is a table showing that the combo-HCV-Ags EIA assays using serum samples provides 100% sensitivity and 100% specificity in 121 serum specimens tested, including 38 negative for serum HCV RNA by PCR and 83 positive for serum HCV RNA by PCR ranging from 94 IU/mL to 14,400,000 IU/mL.

FIG. 7B shows the HCV-Ags level in serum determined by optical density of the combo HCV-Ags EIA assay was significantly correlated to serum HCV RNA level determined by routine HCV RNA PCR (r²=0.812, p<0.01).

FIG. 8A is a table showing that the combo-HCV-Ags EIA assays using urine samples provides 98.7% sensitivity and 100% specificity in 100 urine specimens tested, including 20 negative for serum HCV RNA by PCR and 80 positive for serum HCV RNA by PCR ranging from 62,000 IU/mL to 9,960,000 IU/mL. Of the 100 subjects tested, the one false negative resulted from a subject with End Stage Renal Diseases (ESRD) on hemodialysis (HD).

FIG. 8B shows the HCV-Ags level in urine sample determined by optical density of the combo HCV-Ags EIA assay was significantly correlated to serum HCV RNA level determined by routine HCV RNA PCR (r²=0.821, p<0.01).

FIG. 9 is a table summarizing the combo HCV-Ags EIA testing results for serum specimens (top 2 rows) and urine specimens (bottom 2 rows) in 15 individuals positive for serum anti-HCV, but negative for serum HCV RNA by PCR. As set forth in the table, after denaturation (Method I), 6/15 (40%) of these serum specimens showed positive results, but when the denaturation step was omitted (Method II), all (100%) of these same specimens showed negative results. These results indicate that denaturing serum specimens release IC-HCV antigens from IC-HCV complexes and result in false positive test results in these individuals. However, denaturation of the urine specimens from the same 15 individuals resulted in 100% negative test results, fully consistent to non-denaturation of the same specimens. Thus, denaturation or not will not impact the combo HCV-Ags EIA testing results using urine specimens.

FIG. 10A is a bar graph showing the unexpected increase in sensitivity of the combo-HCV-Ags EIA assay resulting from mixing the test (serum) sample and the detection antibodies according to Example 5.5. NC: negative control using a serum specimen negative for anti-HCV and HCV RNA by PCR; 2 Core Abs: using 2 kinds of anti-HCVcAg antibodies for EIA; Combo: the combo HCV-Ags EIA (Example 2). Based on measured OD values, mixing the test sample with the detection antibodies prior to contact with the assay substrate having the capture antibodies thereon provides an improvement in sensitivity by about 27% compared to combo HCV-Ags EIA without mixing; whereas, the mixing step according to Example 5.5 improved the sensitivity of 2 core EIA by 8%, compared to the same method without the mixing step.

FIG. 10B is a bar graph showing the detection of HCV-Ags with the mixing step described in FIG. 10A in serially diluted serum specimens from 2 subjects with positive serum HCV RNA by PCR using a combo-HCV-Ags EIA assay. The samples and detection antibodies were mixed together prior to contact with the assay substrate having the capture antibodies thereon. NC: negative control, a serum specimen from a subject negative for anti-HCV and HCV RNA by PCR. Denaturing method has higher ODs, but both methods have comparable detection limit, e.g., equivalent to serum HCV RNA 140 IU/mL.

FIG. 10C is a bar graph showing the detection of HCV-Ags with the mixing step described in FIG. 10A in urine specimens from 5 subjects with positive and low serum HCV RNA by PCR using combo-HCV-Ags EIA assay. The samples and detection antibodies were mixed together prior to contact with the assay substrate having the capture antibodies thereon. NC: negative control, a urine specimen from a subject negative for anti-HCV and serum HCV RNA by PCR. The OD values indicated detection limit was equivalent to serum HCV RNA 63-94 IU/mL.

FIG. 10D is a graph showing that results from combo HCV-Ags EIA tests from 2 serum samples from subjects clinically diagnosed with acute hepatitis C virus infection, i.e., subjects testing positive for HCV RNA by PCR, but negative for Anti-HCV antibodies. PBS: PBS negative control; NC: negative control from a serum specimen negative for anti-HCV and HCV RNA by RT PCR. S1 and S2: tested serum samples 1 and 2 with acute HCV infection. The 51 and S2 graphs indicated positive test results at 59 days (S2) and 65 days (S1) before anti-HCV test became positive using a combo HCV-Ags EIA test. Thus, combo HCV-Ags assays according to the present invention are able to detect acute hepatitis virus infection before the appearance of anti-HCV antibodies. Thus, the detection of free hepatitis virus antigen according to the present invention is independent of a subject's development and/or presence of an immune response, e.g., antibody formation.

FIG. 11A are pictures of test strips of LFT assays. Panel A) Using HCVcAg alone in LFT test strips could not detect any positive signal; Panel B) Using combo-HCV-Ags LFT test strips, the specific signal was detectable in serum samples, and Panel 3) Using combo-HCV-Ags LFT test strips, the specific signal was detectable in urine samples. These also confirmed the presence of HCV NS3, NS4b, and NS5a antigens besides HCVcAg in both serum and urine samples during HCV infection. Samples from subjects with active HCV infection was confirmed by serum HCV RNA using PCR. In each panel, strip 1: PBS buffer as negative control; strip 2: samples testing positive for anti-HCV, but negative for HCV RNA as negative control; strips 3-5 actively HCV-infected samples.

FIG. 11B are pictures of test strips of combo-HCV-Ags LFT assays using urine samples showing that adding an additional mAb to HCVcAg to the combo-HCV-Ags LFT test strip further increases sensitivity. Panel A shows test strips using 1 HCVcAg mAb+antibodies against NS3, NS4b, and NS5a; Panel B shows test strips using 2 different HCVcAg mAbs+antibodies against NS3, NS4b, and NS5a. In each panel, strip C=negative control, urine sample with negative anti-HCV and serum HCV RNA by PCR; and strips 1-5 are HCV genotypes 1, 2, 3, 4, and 6, respectively. Similar results were shown with GT-5 (not shown).

FIG. 12A are pictures of the test strips using combo-HCV-Ags LFT assays showing that mixing the test serum samples with the colloid gold solution (detector antibodies conjugated with colloid gold) before adding to the test pad, in accordance with Example 6.3, significantly improved the sensitivity of the combo-HCV-Ags LFT assays according to the present invention for serum test samples.

FIG. 12B shows the test results using the combo-HCV-Ags LFT assays with the mixing step according to Example 6.3 for 5 serum specimens with low serum HCV RNA level by PCR, the lowest detection limit of the combo HCV-Ags LFT is in the range equivalent to serum HCV RNA level of 26-63 IU/mL. C: negative control with a serum specimen negative for anti-HCV and serum HCV RNA by PCR.

FIG. 12C is a table showing that the combo-HCV-Ags LFT assays using serum samples provides 100% sensitivity and 100% specificity in 60 serum specimens tested, including 20 negative for serum HCV RNA by PCR and 40 positive for serum HCV RNA by PCR ranging from 240 IU/mL to 1,740,000 IU/mL.

FIG. 13A are pictures of the test strips using combo-HCV-Ags LFT assays showing that mixing the test urine samples with the colloid gold solution (detector antibodies conjugated with colloid gold) before adding to the test pad significantly improved the sensitivity of the combo-HCV-Ags LFT assays according to the present invention for urine samples.

FIG. 13B shows the test results using the combo-HCV-Ags LFT assays the combo-HCV-Ags LFT assays for 5 urine specimens with low serum HCV RNA by PCR with the lowest detection limit of the HCV-Ags LFT in urine specimens is in the range equivalent to serum HCV RNA level of 63-94 IU/mL. C: negative control with a urine specimen negative for anti-HCV and serum HCV RNA by PCR.

FIG. 13C is a table showing that the combo-HCV-Ags EIA assays using urine samples provides 100% sensitivity and 100% specificity in 93 urine specimens tested, including 15 negative for serum HCV RNA by PCR and 78 positive for serum HCV RNA by PCR ranging from 570 IU/mL to 9,960,000 IU/mL.

FIG. 14A is a graph showing the OD values of the combo HBsAg EIA assay with the mixing step according to Example 5.5 using urine specimens from 5 subjects positive for serum HBsAg (#1-5) and a negative control (#6). For samples 1-5, the first bars are the results using one antibody against HBsAg and the second bars are the results using two different antibodies against HBsAg. Neg Control, a urine specimen with negative serum HBsAg test.

FIG. 14B are LFT test strips of urine samples with the mixing step according to Example 6.3 from 6 subjects with positive serum HBsAg using one antibody against HBsAg (strips labeled with “a”) and using two different antibodies against HBsAg (strips labeled with “b”). NC, a negative control urine specimen with negative serum HBsAg test.

### DETAILED DESCRIPTION OF THE INVENTION

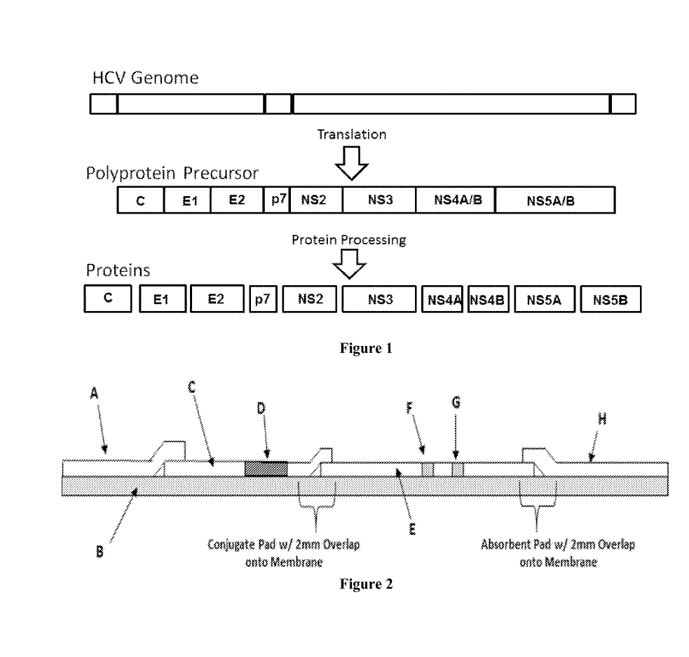

As shown in FIG. 1, during HCV replication and life cycle, a large HCV precursor protein is translated and used to produce various HCV proteins, including structural (envelop and core proteins) and non-structural (NS) proteins. Some of these proteins are highly conserved and antigenic, as their corresponding antibodies are detectable in subjects having been infected with HCV even after clearance of the infection.

As disclosed herein, in addition to HCV Core Antigen (HCVcAg), several other HCV proteins including highly conserved HCV non-structural (HCV NS) proteins, e.g., NS3, NS4b, and NS5a, are found to be continually expressed as free HCV antigens in the blood and urine of subjects having active HCV infections, e.g., subjects testing positive for serum HCV RNA using PCR. As used herein, “free antigens” refers to antigens that have yet to become a part of a subject's native immune complex, e.g., not yet bound by antibodies created by the given subject's immune response. The term “free hepatitis virus antigens” refers to hepatitis virus antigens that have yet to become part of a subject's immune complex. The term “free HCV antigens” refers to HCV antigens that have yet to become part of a subject's immune complex. As used herein, “IC complex” refers to a complex between an antigen and one or more antibodies resulting from a subject's immune response. An “IC-HCV complex” refers to an HCV antigen immune complex between an HCV antigen and one or more antibodies resulting from a subject's immune response. For the ease of convenience, as disclosed herein, HCV antigens that are and/or were part of a subject's immune complex will be designated as “IC-HCV antigens”. IC-HCV antigens include HCV antigens that are still part of an IC complex and those that have become unbound (or released) from IC-HCV complexes by non-natural conditions, e.g., laboratory assays which denature the IC-HCV complexes. As used herein, “total HCV antigens” refers to the total of any free HCV antigens plus the total of any IC-HCV antigens. IC-HCV antigens may be found in samples (except urine) from subjects having active HCV infections (i.e., testing positive for HCV RNA using PCR) and subjects having had past, but cleared, HCV infections (i.e., testing negative for HCV RNA using PCR). Free HCV antigens are found in samples (including both blood and urine) from subjects having active HCV infections.

The present invention is directed to assays, systems, and kits for detecting the presence of a plurality of hepatitis virus antigens as free hepatitis virus antigens and/or IC-hepatitis virus antigens in a sample. In some embodiments, the present invention is directed to assays, systems, and kits for detecting the presence of a plurality of free HCV antigens and/or a plurality of IC-HCV antigens in a sample. In some embodiments, the HCV antigens are simultaneously detected and/or detected in the same assay step. The sample may be a biological sample such as whole blood, serum, plasma, urine, or other body fluids or tissues in which free HCV antigens and/or IC-HCV antigens can be found, or a synthetic sample, e.g., a laboratory made sample used for control experiments. In some embodiments, the sample is a urine sample.

Suitable HCV antigens include HCVcAg, HCV E1, E2, NS2, NS3, NS4a, NS4b, NS5a, and NS5b proteins. In some embodiments, a plurality of total HCV antigens (i.e., free HCV antigens plus IC-HCV antigens) are detected. The HCV antigens of the plurality may be free HCV antigens and/or IC-HCV antigens. In some embodiments, a plurality of only free HCV antigens are detected. As used herein, references to specific HCV antigens of “a plurality of free HCV antigens”, even without specifically designating each HCV antigen as being a free HCV antigen, means that each referenced HCV antigen of the plurality is a free HCV antigen. In some embodiments, the plurality of free or total HCV antigens include HCVcAg. In some embodiments, the plurality of free or total HCV antigens comprise HCVcAg and one or more of E1, E2, NS2, NS3, NS4a, NS4b, NS5a, and NS5b proteins. In some embodiments, the plurality of free or total HCV antigens comprise HCVcAg, NS3, NS4b, and NS5a proteins. In some embodiments, the plurality of free or total HCV antigens consist of HCVcAg, HCV NS3, NS4b, and NS5a proteins.

As provided herein, usage of the term “combo-HCV-Ags” in conjunction with assay, system, or kit, refers to an assay, system, or kit according to the present invention (e.g., assays in which a plurality of free or total HCV antigens are detected). Thus, for example, a “combo-HCV-Ags assay” refers to an assay in which a plurality of free or total HCV antigens are detected. The assay platform of the assays of the present invention may be any immunoassay, including enzyme immune assays (EIAs), microplate-based immunoassays (MIAs), chemiluminescent immunoassays (CIAs), fluorescent immunoassays (FIA), enzyme-linked immunosorbent assays (ELISAs), or lateral flow tests (LFTs) known in the art, and may be automated or manual. The various assays may employ any suitable labeling and detection system. As used herein, a “detectable label” is a compound or composition that is detectable by spectroscopic, photochemical, biochemical, immunochemical, or chemical means. The use of “labeled” to modify a substance, e.g., a labeled antibody, means that the substance has a detectable label added thereto. A substance, e.g., antibody, having a detectable label means that a detectable label that is not normally linked or conjugated to the substance has been linked or conjugated to the substance by the hand of man. As used herein, the phrase “by the hand of man” means that a person or an object under the direction of a person (e.g., a robot or a machine operated or programmed by a person), not nature itself, has performed the specified act. Thus, the steps set forth in the claims are performed by the hand of man, e.g., a person or an object under the direction of the person.

As disclosed herein, combo-HCV-Ags assays according to the present invention result in a significant improvement in sensitivity over assays in which only one HCV antigen is detected. Therefore, in some embodiments, the present invention is directed to assays, systems, and kits for diagnosing a subject as having or having had an HCV infection, which comprises detecting the presence (or absence) of a plurality of HCV antigens in a sample obtained from the subject, and diagnosing the subject as having or having had an HCV infection where the plurality of HCV antigens are present.

Additionally, as disclosed herein, subjecting the samples to be tested to conditions, e.g., denaturing conditions, which disassociate IC-HCV complexes results in increased assay sensitivity. In fact, as shown herein, subjecting the test samples to denaturing conditions before detection results in 100% sensitivity. Based on the results of the experiments below, it is believed that IC-HCV antigens that are still part of the immune complex are not readily detected or bound by additional antibodies, e.g., detection antibodies, added thereto possibly because the binding sites are occupied by the antibodies of the immune complex. Therefore, in some embodiments, a sample to be tested is subjected a denaturing condition that disassociates the IC-HCV complexes prior to detection. Such denaturing conditions include pre-treatment of serum specimens with a denaturing solution having about 0.3% Triton X-100, about 1.5% 3-[(3-Cholamidopropyl) dimethylammonio]-1-propanesulfonate (CHAPS), and about 15% sodium dodecyl sulfate (SDS), pH of about 8.5, at about 56° C. for about 30 minutes. Conditions that result in similar effects may be readily determined by those skilled in the art and are contemplated herein.

As disclosed herein, HCV antigens may remain present as IC-HCV complexes in the blood of subjects who cleared an HCV infection, e.g., subjects who test positive for anti-HCV antibodies and test negative for HCV RNA using PCR. Thus, subjecting a sample that may have IC-HCV complexes to denaturing conditions prior to detection may lead to false positives for active HCV infections in subjects having had past, but cleared, HCV infections. Therefore, in some embodiments, to avoid detecting IC-HCV antigens, the methods of the present invention do not subject the sample being tested to denaturing conditions that disassociate the HCV antigens from IC-HCV complexes prior to detection. In other words, where the detection of denatured IC-HCV antigens is to be avoided, the methods of the present invention exclude denaturing IC-HCV complexes that may be present in the sample prior to detection. Or, said another way, such methods of the present invention avoid conditions that disassociate the HCV antigens from IC-HCV complexes prior to detection.

As disclosed herein, the detection of a plurality of free HCV antigens in a sample that has not been subjected to denaturing conditions (e.g., conditions that disassociate IC complexes) using a combo-HCV-Ags assay according to the present invention results in the detection of active HCV infection at a level that is equivalent to serum HCV RNA 140 IU/mL using HCV RNA PCR. In other words, the combo-HCV-Ags assays of the present invention, which exclude denaturing conditions, have a sensitivity and specificity for detecting active HCV infections that is comparable to PCR assays for serum HCV RNA. Therefore, in some embodiments, the present invention is directed to assays, systems, and kits for diagnosing a subject as having an active HCV infection, which comprises detecting the presence (or absence) of a plurality of free HCV antigens in a sample, which has not been subjected to denaturing conditions, obtained from the subject, and diagnosing the subject as having an active HCV infection where the plurality of free HCV antigens are present.

As disclosed herein, HCV antigens are detectable in urine samples of subjects testing positive for serum HCV RNA using PCR, but are not detectable in urine samples of subjects testing negative for serum HCV RNA using PCR. Additionally, IC-HCV complexes are not present in urine samples as denaturing the urine samples before detection does not result in urine samples from subjects having had past, but cleared, HCV infections, to test positive for any HCV antigens (free or total HCV antigens). Thus, in some embodiments, a urine sample from a subject may be used to detect the presence or absence of free HCV antigens only. The subject can then be diagnosed as having an active HCV infection where the presence of free HCV antigens in the urine sample is detected or diagnosed as not having an active HCV infection where free HCV antigens are not detected in the urine sample.

As shown herein, the use of two different antibodies to detect a given single antigen results in an unexpected superior increase in assay sensitivity and specificity. Therefore, in some embodiments, the present invention provides assays, systems, and kits for detecting a hepatitis virus antigen, which comprises using two or more different antibodies against the same hepatitis virus antigen. In some embodiments, the assays, systems, and kits, which employ two or more different antibodies against the same hepatitis virus antigen, is a combo-HCV-Ags assay, system, or kit as disclosed herein.

As shown herein, when the detection antibodies against the plurality of HCV antigens are mixed together and incubated with the sample to be tested, the sensitivities of the combo-HCV-Ags assays were significantly increased. Therefore, in some embodiments, the present invention provides immunoassays wherein the test sample and the detection antibodies are mixed together before being contacted with the assay substrate having capture antibodies coated or immobilized thereon.

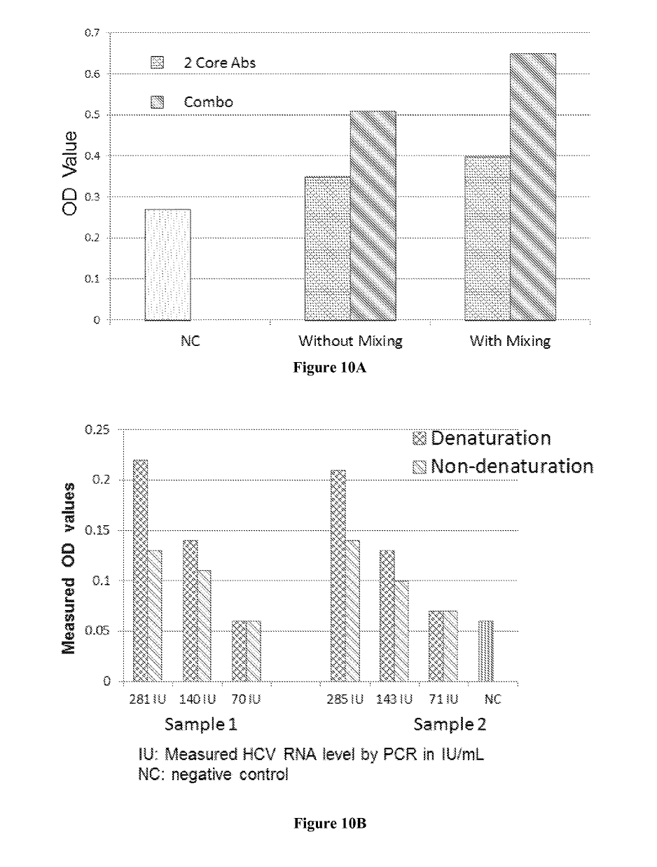

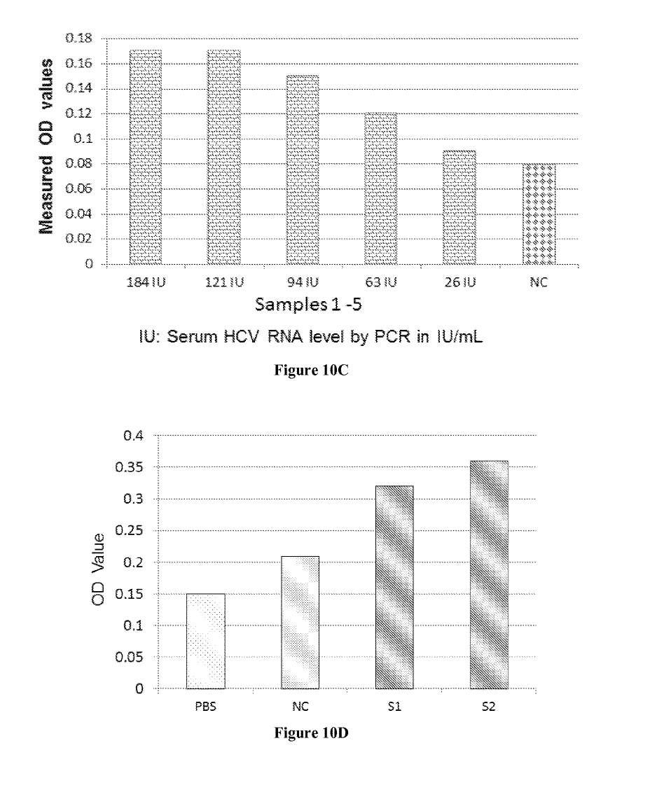

As shown in FIG. 10D, the detection of free hepatitis virus antigen(s) is independent of a subject's development and/or presence of an immune response, e.g., antibody formation. Unlike assays that detect or measure antibodies against hepatitis virus antigens, combo HCV-Ags assays according to the present invention may be used to detect acute hepatitis virus infection before the appearance of anti-HCV antibodies. Thus, in some embodiments, the present invention is directed to methods of detecting acute hepatitis virus infection in a subject, diagnosing the subject as having an acute hepatitis virus infection, or both, before the subject develops antibodies against the hepatitis virus.

The sensitivity and specificity of the assays, systems, and kits according to the present invention can be further improved by optimizing the assay conditions, e.g., reaction times and temperatures, and/or modifying or substituting the reagents, e.g., using a different detection and labeling system, employed using methods known in the art.

In summary, the present invention provides immuno-based assays, systems, and kits for one or more hepatitis virus antigens wherein (1) two or more antibodies against a given single hepatitis virus antigen are used at the same time in the same detection step, (2) a plurality of hepatitis virus antigens are detected at the same time in the same detection step, (3) the detection antibodies against the hepatitis virus antigen(s) are mixed with the sample prior to contact with the assay substrate having the capture antibodies, (4) only free hepatitis virus antigen(s) is/are detected, and/or (5) total hepatitis virus antigen is detected.

In addition to being used for scientific and clinical research, the assays and systems of the present invention may be used to (1) screen for the presence of free hepatitis virus antigen(s) in a subject, the presence of which can be used to diagnose the subject as having an active hepatitis virus infection; (2) distinguish ongoing active hepatitis virus infection from resolved past HCV infection/exposure using a simple one test approach without the need for a confirmatory PCR test for hepatitis virus RNA; (3) identify active hepatitis virus infection in the early stage of infection, e.g., the pre-seroconversion window period, characterized by the presence of free hepatitis virus antigen, and the absence of anti-hepatitis virus antibodies and/or hepatitis virus antigen immune complexes; (4) identify hepatitis virus infection in individuals who are immunocompromised and unable to produce anti-hepatitis virus antibodies, such as subjects on immunosuppressive treatment or hemodialysis; (5) monitor hepatitis virus RNA levels, e.g., use the amount of free hepatitis virus antigens as an indication of hepatitis virus RNA levels; and (6) monitor the effect of treatments on hepatitis virus infections in subjects.

Since some embodiments of the present invention can be performed by a single step, e.g., loading a test sample on a test pad of an LFT test strip in order to detect the presence of hepatitis virus antigen(s), such assays, systems, and kits are cost-effective, convenient, time-saving, affordable, and can readily be performed in a laboratory or at the point-of-care by physicians in a clinic and by subjects at home.

The following examples are intended to illustrate, but not to limit the invention.

### EXAMPLES

### Materials

The monoclonal antibodies (mAb) and polyclonal antibodies (pAb) exemplified in the experiments herein are set forth in the Table A as follows:

TABLE A

| Hepatitis Antigen Antibody | Cat. No. | Clone No. | Sequence Raised/Specific Against |
|---|---|---|---|
| Anti-HCVcAg mAb | ab2740 | C7-50 | 21-40 aa |
| Anti-HCVcAg mAb | ab18929 | 11-B3 | 70-90 aa |
| Anti-NS3 mAb | ab65407 | 8G-2 | 1340-1470 aa |
| Anti-NS4b mAb | ab24283 | 2-H1 | 1710-1730 aa |
| Anti-NS5a mAb | ab13833 | H26 | Recombinant full length NS5a |
| Anti-NS4 pAb | ab20955 | | Recombinant NS4 |
| Anti-NS3 pAb | ab21124 | | Recombinant full length NS3 |
| Anti-HCVcAg pAb | ab50288 | | Recombinant full length HCVcAg |
| Anti-NS5a pAb | MBS630668 | | Recombinant corresponding to NS5a + b |
| Anti-HBV antigen HBsAg antibody | SAB4700767 | HB5 | Purified HbsAg |
| Anti-HBV antigen HBsAg antibody | SAB4700768 | HB3 | Purified HbsAg |
| Anti-HCVcAg mAb* | sc-57800 | C7-50 | 21-40 aa |
| Anti-NS3 mAb* | sc-52805 | 12-5 | Recombinant corresponding to at least 60 aa of |
| | | | NS3 |
| Anti-NS4b mAb* | sc-52416 | 2-H1 | Recombinant NS4b |
| Anti-NS5a mAb* | sc-65458 | 1877 | Recombinant full length NS5a |

Antibodies having catalogue numbers starting with “ab” are from Abcam, Inc. Cambridge, MA; and “MB” are from Mybiosource; San Diego, CA; “SAB” are from Sigma-Aldrich, Inc., Saint Louis, MO; and “sc” are from Santa Cruz Biotechnology, Inc., Dallas, TX.
*The HCV antibodies that were not used in the experiments herein.

### Methods

### Example 1—HCV RNA PCR

As disclosed herein, the presence of serum HCV RNA was assayed using polymerase chain reaction (PCR) methods known in the art. Specifically, serum HCV RNA was quantitated by real-time polymerase chain reaction (PCR) using Roche COBAS® AmpliPrep/COBAS® TaqMan® HCV assay, which has a lower detection limit of 43 IU/mL and quantitative limit of 100 IU/mL (Roche Molecular Diagnostics, Pleasanton, Calif.); or using Abbott RealTime HCV assay, which has both lower detection limit and quantitative limit of 12 IU/mL (Abbott Laboratories, Abbott Park, Ill.).

### Example 2—EIA Protocol

The following protocol was used in the EIA experiments disclosed herein unless indicated otherwise.

Step 1. Coating of the Assay Substrate. A 96-well PVC microtiter plate was used as the assay substrate, however, other substrates, e.g., assay beads, known in the art may be used. Each test well of the microtiter plate was coated with a sufficient amount, 50-200 μL, e.g., about 100 μL, of capture antibodies diluted with carbonate/bicarbonate buffer (pH about 7.0-9.5, e.g., about 9.0). The capture antibodies were mixture of monoclonal antibodies against HCVcAg-1 (about 5-20 μg/mL, e.g., about 10 μg/mL), HCVcAg-2 (about 5-20 μg/mL, e.g., about 10 μg/mL), NS3 (about 5-20 μg/mL, e.g., about 5 μg/mL), NS4b (about 5-20 μg/mL, e.g., about 5 μg/mL), and NS5a (about 5-20 μg/mL, e.g., about 5 μg/mL).

Step 2. Incubation. The microtiter plate from Step 1 was covered and incubated at 4° C. for overnight (or 37° C. for about 15-120 minutes, e.g., about 60 minutes).

Step 3. Washing. After Step 2, the microtiter plate was washed for 3 times by filling each well with about 100-400 μL, e.g., about 300 μL, of TBS-T solution (wash solution) and flicking the plate over a sink. The remaining wash solution was then removed by patting the plate with a paper towel.

Step 4. Blocking Non-specific Binding. Each well was then treated by adding about 150-300 μL, e.g., about 300 μL, of blocking buffer containing about 1-5%, e.g., about 3%, BSA. The plate was then incubated at room temperature for about 15-90 minutes, e.g., about 60 minutes, to block the remaining protein-binding sites in the coated wells.

Optional Step 5. Pretreatment of Test Samples. When performed, e.g., on serum or plasma samples, about 25-150 μL, e.g., about 100 μL, of the test sample was mixed with about 50-200 μL, e.g., about 50 μL, of pretreatment solution (0.3% Triton X-100, 1.5% 3-[(3-Cholamidopropyl) dimethylammonio]-1-propanesulfonate (CHAPS) and 15% sodium dodecyl sulfate (SDS)), in a 1.5 mL centrifuge tube, and then incubated at 56° C. for about 30-60 minutes, e.g., about 60 minutes.

Step 6. Serum/plasma or Urine Specimen Loading. After removing the blocking buffer from each well of the microtiter plate, about 50-300 μL, e.g., about 100 μL, of the pretreated serum or plasma sample, or about 50-300 μL, e.g., about 100 μL, of untreated urine sample were added to each well. The plate was then covered and incubated, under gentle agitation, at room temperature for about 30-120 minutes, e.g., about 90 minutes.

Step 7. Washing. Then the wells were washed 3 times by filling each well with about 150-300 μL, e.g., about 300 μL, of TBS-T solution and flicking the plate over a sink. The remaining wash solution was then removed by patting the plate with a paper towel.

Step 8. Loading the Detection Antibodies. After Step 7, about 50-300 μL, e.g., about 100 μL, of detection antibodies were added to each test well. For the combo-HCV-Ags assays according to the present invention, the first detection antibodies were mixture of polyclonal antibodies against HCVcAg, NS3, NS4b, and NS5a. For assays according to the prior art, the detection antibodies consisted only of anti-HCVcAg antibodies. Then the microtiter plate was covered and incubated, under gentle agitation, at room temperature for about 30-120 minutes, e.g., about 90 minutes.

Step 9. Washing. To remove unbound detection antibodies, the wells were washed 3 times by filling each well with about 150-300 μL, e.g., about 300 μL, of TBS-T solution and flicking the plate over a sink. The remaining wash solution was then removed by patting the plate with a paper towel.

Step 10. Loading HRP-Conjugated Antibodies. About 50-300 μL, e.g., about 100 μL, of HRP-conjugated IgG antibodies specific against the species of the detection antibodies were added to each test well. The HRP-conjugated IgG antibodies were diluted to a concentration at 1:3000-1:5000, e.g., about 1:4000 dilution in blocking buffer immediately before use. The plate was then covered and incubated at room temperature for about 15-90 minutes, e.g., about 30 minutes.

Step 11. Washing. To remove unbound antibodies, the wells were washed 3 times by filling each well with about 150-300 μL, e.g., about 300 μL, of TBS-T solution and flicking the plate over a sink. The remaining wash solution was then removed by patting the plate with a paper towel.

Step 12. Color Reaction. About 50-300 μL, e.g., about 100 μL, of Substrate Solution (OPD) was added to each test well and incubated at room temperature for about 5-30, usually about 15 minutes, followed by about 25-50 μL, e.g., about 50 μL, of stop solution to stop the enzymatic reaction.

Optical density was measured using 450 nm as the primary wavelength on an ELX 800 Universal Microplate Reader (Bio-TEK Instruments, Inc., Winooski, Vt.). The cut off value was determined by the mean negative OD value plus 3×standard deviation (SD). The test results were considered positive, if the tested OD value was >the cut off value; the test results were considered negative, if the tested OD value was <the cut off value; and the test results were considered equivocal, if the tested OD value was=the cut off value.

### Example 3—LET Protocol

### A. Test Strips

As shown in FIG. 2, the LFT test strips exemplified in the experiments herein comprise the following components: A=Sample Pad, B=Backing card, C=Conjugate pad, D=Capture antibody conjugated with colloid gold particles, E=Nitrocellulose membrane, F=Test line, G=Control line, H=Absorbent pad. The test strips were constructed as follows unless indicated otherwise. However, other test strips, dipsticks, etc. known in the art may be used in accordance with the present invention. Thus, the term “test strips” is herein to generically refer to assay substrates, used for LFT assays, having sample pad where a test sample is loaded and then flows through a test line and a control line as having capture antibodies as described below. Although the LFT experiments herein exemplify the use of a colloid gold labeling system, other labeling systems known in the art may be used.

Step 1. Colloid Gold Conjugation of the Detector Antibodies. The pH value of the colloid gold solution to be used was adjusted to about 7.4-9.0, e.g., about 8.5, with 0.2 M potassium carbonate. For assays detecting only HCVcAg by itself, a monoclonal antibody against HCVcAg was mixed with the colloid gold solution in a total volume of 5.4 mL to give a concentration of about 5-20 μg/mL, e.g., about 10 μg/mL. For the combo-HCV-Ags assays according to the present invention, monoclonal or polyclonal IgG antibodies against HCVcAg-1, HCVcAg-2, NS3, Ns4b, and NS5a were then mixed with the colloid gold solution in a total volume of 5.4 mL. The concentration of each of these anti HCV antibodies was about 5-20 μg/mL, e.g., about 10 μg/mL. After the mixture was stirred vigorously for about 15-60 minutes, e.g., about 30 minutes at room temperature, 0.6 mL of about 5-20%, e.g., about 10%, BSA (pH 9.0) was added to block excess reactivity of the gold colloid. Then the mixture was stirred about 15-60 minutes, e.g., about 30 minutes, at room temperature. The mixture was then centrifuged at 12,000 rpm at 4° C. for about 15-60 minutes, e.g., about 30 minutes, and the resulting conjugated pellet was re-suspended and wash 2 times with 2 mM borax buffer (pH 9.0, containing about 1-5%, e.g., about 1%, BSA). After 1:100 dilution of the conjugate, the OD value was adjusted to reach 10±0.5 at a wavelength of 540 nM. The pellet was re-suspended in borax buffer (about 1-5 mM, e.g., about 2 mM, pH 9.0, containing 20% sucrose, and about 1-5%, e.g., about 1%, BSA) and kept at 4° C. until use.

Step 2. Treatment of the Conjugate Pad. The conjugate pad (FIG. 2 “C”) was treated with about 10-50 mM, e.g., about 20 mM, phosphate buffer containing about 1-5%, e.g., about 3%, BSA, about 0.5-5%, e.g., about 1%, Tween 20, about 0.1-1.5%, e.g., about 0.3%, polyvinylpyrrolidone K30, and about 0.02% sodium azide (pH of about 7.0-8.0, e.g., about 7.4) for about 5-60 minutes, e.g., about 10 minutes, at room temperature, then dried at 37° C. for about 15-60 minutes, e.g., about 30 minutes.

Step 3. Loading Detector Antibodies Conjugated with Colloid Gold to the Treated Conjugate Pad. As shown in FIG. 2 “D”, the HCV-Ags specific detector antibodies conjugated with colloid gold particles in Step 1 were then dispensed to the treated conjugate pad (prepared in Step 2) at a rate of about 10-30 μL/cm, e.g., about 10 μL/cm, using a BioDot XYZ platform (BioDot, Irvine, Calif.), then dried at 37° C. for about 15-60 minutes, e.g., about 30 minutes.

Step 4. Loading Capture Antibodies Specific for HCV Antigens to the Test Line. About 0.5-2 mg/mL, e.g., about 1 mg/mL, of capture antibodies were dispensed to the test line on the nitrocellulose membrane at a rate of about 0.1-2 μL/cm, e.g., about 0.9 μL/cm and speed of about 2-10 cm/sec, e.g., about 4 cm/second. For the combo-HCV-Ags assays according to the present invention, the capture antibodies were a mixture of polyclonal capture monoclonal or polyclonal antibodies specific to HCVcAg, NS3, NS4b, and NS5a. For the assays detecting only HCVcAg, the capture antibodies consisted of only antibodies against HCVcAg.

Step 5. Loading Capture Antibody Specific for mouse IgG to the Control Line. As shown in FIG. 2 “G”, polyclonal capture antibody specific to mouse IgG, at about 0.5-2 mg/mL, e.g., about 1 mg/mL, was dispensed to the control line on the nitrocellulose membrane at a rate of about 0.1-2 μL/cm, e.g., about 0.9 μL/cm, and speed of about 2-10 cm/sec, e.g., about 4 cm/sec, then dried at 37° C. for about 15-90 minutes, e.g., about 30 minutes.

Step 6. Assembling Lateral Flow Strip. The absorbent pad (FIG. 2 “H”), nitrocellulose membrane (treated as in Steps 4 and 5, FIG. 2 “E”), conjugate pad (treated as in Step 3, FIG. 2 “C”), and sample pad (FIG. 2 “A”) are then assembled as a strip on backing card (FIG. 2 “B:), that is then attached to a plastic scale board with about a 1- to 2-mm overlap, sequentially. The assembled plate was cut into about 2-10 mm, e.g., about 3 mm, wide pieces using a CM 4000 cutter (BioDot, Irvine, Calif.). The generated test strips were packaged in a plastic bag with desiccant and stored at 4° C. or room temperature for the experiments.

### B. LFT Assay Protocol

A diluted tested sample (about 100-300 μL, e.g., about 250 μL) or a negative control (e.g., PBS solution, or samples from subjects without HCV infection as determined by PCR) was added to the sample pad and left at room temperature for about 5-45 minutes, usually about 15 minutes.

After the test specimen is loaded to the sample pad (FIG. 2 “A”), it rapidly diffuses into the conjugate pad (FIG. 2 “C”). If the tested sample contains the given HCV antigens, the antigens will react with the HCV detector monoclonal antibodies conjugated to colloidal gold particles and loaded in FIG. 2 “D” area. These HCV Ags/Abs complexes will move along on the nitrocellulose membrane chromatographically via capillary action (FIG. 2 “E”). Eventually, these HCV Ags/Abs complexes will react with the preloaded HCV-specific capture monoclonal or polyclonal antibodies, and be immobilized at the test line area to form a colored band that indicates a positive test result (FIG. 2 “F”). The excessive HCV-specific detector antibodies conjugates (or unreacted, when a tested specimen does not contain HCV-Ags), will migrate along the membrane and be immobilized at the control line area by pre-loaded goat anti-mouse antibody and result in colored band at the control line (FIG. 2 “G”). Therefore, a positive sample will display two bands, one at the test line area and one at the control line area, while a negative sample will show only one band at the control line area.

Thus, if there was a colored line at both the test line and control line, the test sample was deemed positive for the given HCV antigen(s). If there was no colored line at the test line area, the test sample was deemed negative for the given HCV antigen(s). However, if there was no colored line at the control line area, the test result was deemed invalid.

### Example 4—Detection of HCV Antigens in Samples

### 4.1 Blood Samples

To determine whether HCV antigens, in addition to HCVcAg, are present in serum samples of subjects having active HCV infections, the following experiment was conducted. Specifically, serum samples obtained from subjects testing positive for serum HCV RNA were tested as set forth in Example 1.

Western blots show that HCVcAg, NS3, NS4b, and NS5a are present in serum samples of subjects having an active HCV infection for all 6 HCV genotypes (data not shown).

### 4.2 Urine Samples

To determine whether HCV antigens are present in urine samples of subjects having active HCV infections, the following experiment was conducted. Specifically, urine samples randomly obtained from subjects who tested positive for serum HCV RNA were collected and stored in −80° C. until use.

Western blots show that HCVcAg, NS3, NS4b, and NS5a are present in urine samples of subjects having an active HCV infection for all 6 HCV genotypes (data not shown). Because IC-HCV complexes are not present in urine, the HCV antigens present in urine are all free HCV antigens.

### Example 5—EIA Experiments

### 5.1 Serum Samples

To determine whether the detection of a plurality of HCV antigens in one sample at the same time is feasible and will provide sufficient sensitivity and specificity for HCV infections, serum and urine samples from subjects testing positive for serum HCV RNA were tested using the EIA protocol of Example 2 with Step 5, however for the assays detecting only HCVcAg, only monoclonal antibodies against HCVcAg were coated on the assay substrate, and for the combo-HCV-Ags assays, the capture antibodies coated on the assay substrate included monoclonal antibodies against HCVcAg, NS3, NS4b, and NS5a.

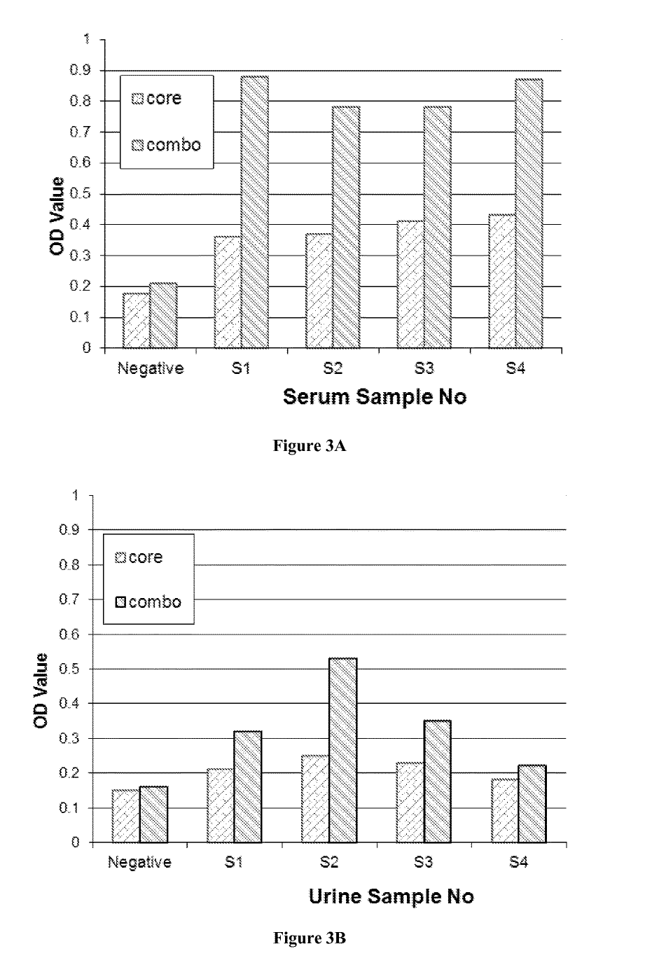

The data provided in FIGS. 3A and 3B show that the combined detection of a plurality of HCV antigens in a single EIA assay system (e.g., combo-HCV-Ags EIA assay) is feasible for serum samples. Additionally, as provided in FIG. 3A, the sensitivity of the combo-HCV-Ags EIA assay is almost double that of the EIA assay where only one HCV antigen, e.g., HCVcAg, is detected.

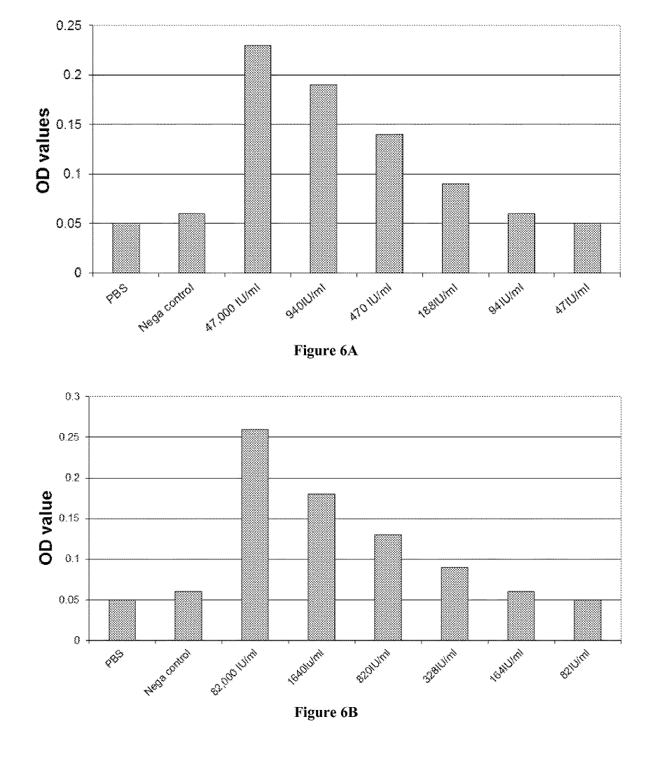

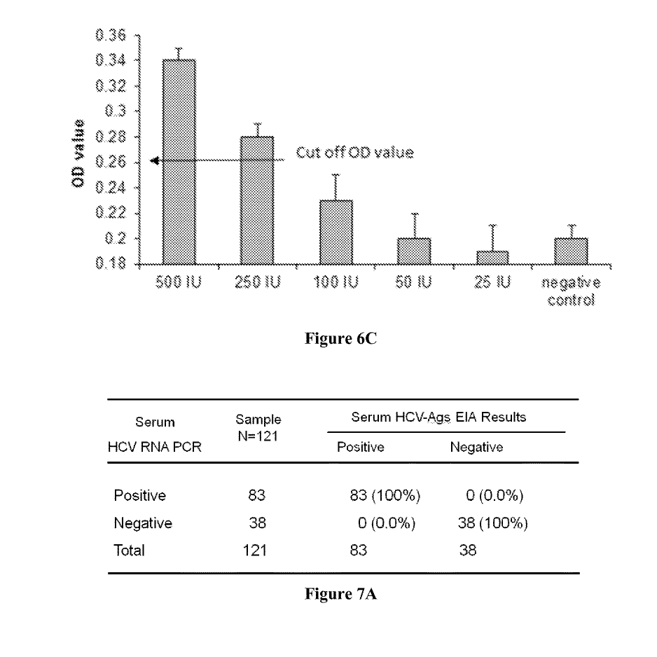

The detection limits of the combo-HCV-Ags EIA assay was determined using the EIA protocol of Example 2 with Step 5 to assay serial dilutions of two serum samples having known amounts of serum HCV RNA (as determined by PCR). The samples were diluted with PBS. Both PBS and a serum sample from a subject testing negative for an active HCV infection (i.e, negative for anti-HCV and HCV RNA by PCR) were used as negative controls. The undiluted serum (control) had baseline HCV RNA 47,000 or 82,000 IU/mL. As shown in FIGS. 6A and 6B, HCV antigens remained detectable at dilution of 1:250, equal to serum HCV RNA equivalent to 188 and 328 IU/mL.

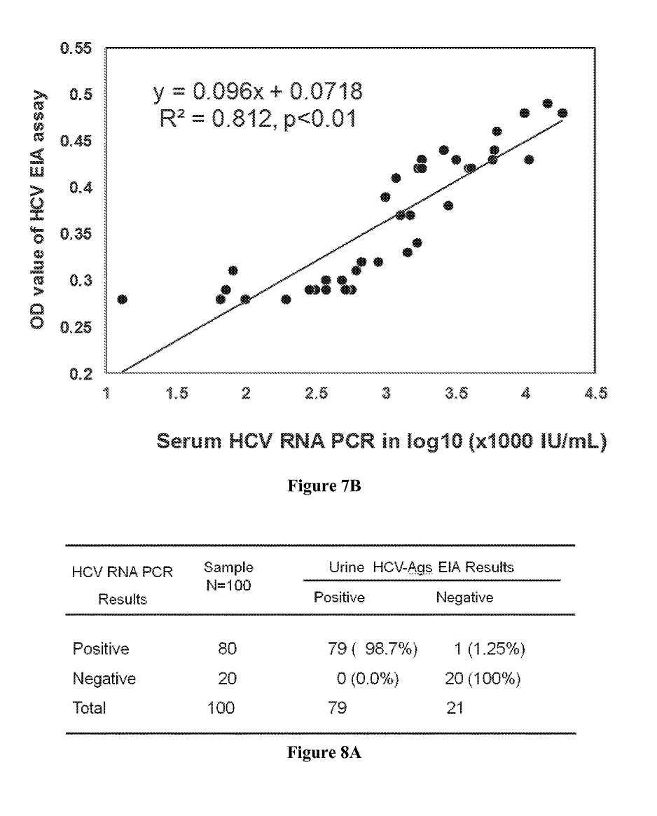

To determine whether the detection limits are independent of HCV genotype, serial dilutions of serum samples having known amounts of serum HCV RNA for each HCV genotype were similarly assayed. FIG. 6C is representative of the results obtained for each of the genotypes and shows that the combo-HCV-Ags EIA assay is capable of a low detection limit that corresponds to a serum HCV RNA level as low as 250 IU/mL and is independent of HCV genotype. FIG. 7A is a table that shows that the combo-HCV-Ags EIA assays using serum samples provides 100% sensitivity and 100% specificity.

As shown in FIG. 7B, the optical density (OD) values determined using the combo-HCV-Ags EIA assay system for serum samples corresponds to serum HCV RNA amounts determined by HCV RNA PCR (r²=0.812, p<0.01).

### 5.2 Urine Samples

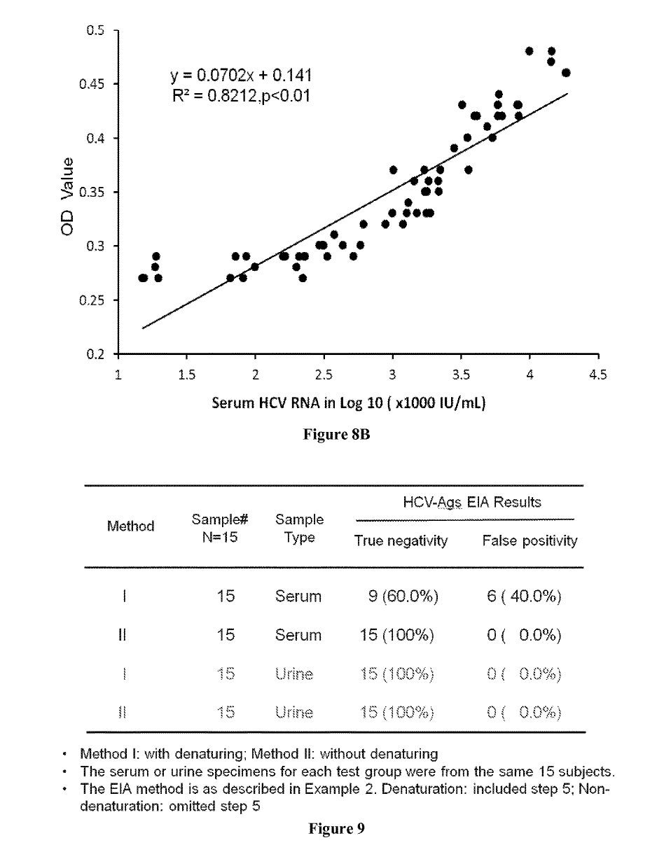

Experiments similar to those set forth in 5.1 above were performed on urine samples except the EIA protocol of Example 2 was performed without Step 5. As set forth in FIG. 8A, the combo-HCV-Ags EIA assay has a 98.7% sensitivity and 100% specificity using urine samples. Of the 100 subjects tested, the one false negative resulted from a subject with End Stage Renal Diseases (ESRD) on hemodialysis (HD). As shown in FIG. 8B, the HCV-Ags level in urine samples determined by optical density of the combo HCV-Ags EIA assay was significantly correlated to serum HCV RNA level determined by HCV RNA PCR (r²=0.821, p<0.01).

### 5.3 Pretreatment of the Serum Test Sample

Serum samples may be pretreated to dissociate HCV antigens from the IC-HCV complex. The results of serum samples from 15 subjects known to be positive for anti-HCV, but negative for serum HCV RNA (e.g., subjects having a past HCV infection and no active HCV infection) were assayed according to Example 2 with Step 5 (Method I, denatured), were compared with the results of serum samples from the same 15 subjects assayed according to Example 2 without Step 5 (Method II, not denatured). As shown in FIG. 9, denaturing the serum samples (Method I) results in positive test results in 6/15 (40%) of the tested serum specimens. These data demonstrated that IC-HCV antigens can be present in the blood of a subject having had a prior, but resolved HCV infection. As these subjects had no active HCV infection, these positive test results are false positives. On the other hand, as shown in FIG. 9, denaturing the urine specimens from the same 15 subjects do not result in such false positive results, as IC-HCV antigens are not present in the urine specimens.

Thus, where the detection of total hepatitis virus antigen(s) is desired (e.g., one need not detect free hepatitis virus antigen(s) only, or distinguish free hepatitis virus antigen(s) from IC-hepatitis virus antigen(s)) the assay sensitivity can be increased by subjecting the sample being tested to denaturing conditions prior to detection. One should note that since urine samples do not contain hepatitis virus immune complexes, subjecting urine samples to denaturing conditions will not increase assay sensitivity.

On the other hand, where the detection of only free hepatitis virus antigen(s) is desired (e.g., IC-hepatitis virus antigens are not to be detected), for example, in order to diagnose a subject as having an active hepatitis virus infection, assay specificity can be increased by testing a sample in which immune complexes are not normally found (e.g., a urine sample) or not subjecting the sample that may contain IC-hepatitis virus antigens to denaturing conditions prior to detection.

In some situations, it may be desired to assay both free hepatitis virus antigen(s) and total hepatitis virus antigen(s) in a subject. For example, in subjects with an ongoing HCV infection, the amount of free HCV antigen(s) compared to the amount of total HCV antigen(s) to differentiate the subject's clinical presentation and monitor the subject's immune response, clinical course, and treatment responses. For example, an increase in the amount of IC-HCV antigens and a decrease in the amount of free HCV antigens in a subject may indicate, for example, a favorable chance of HCV clearance, a decreased chance of liver injury, different responses to HCV treatment, and/or a decreased risk of other clinical complications. On the other hand, a decrease in the amount of IC-HCV antigens and an increase in the amount of free HCV antigens in a subject may indicate, for example, a positive or negative impact on the subject's ability to clear the HCV infection, or the subject's immune system has become compromised.

### 5.4 Detection of HCVcAg Using Two Different Antibodies

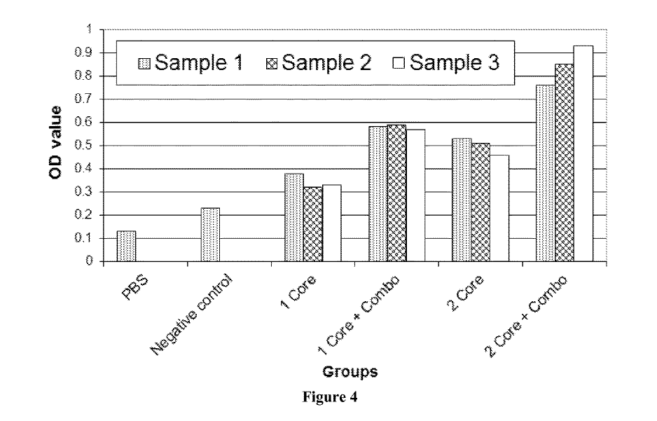

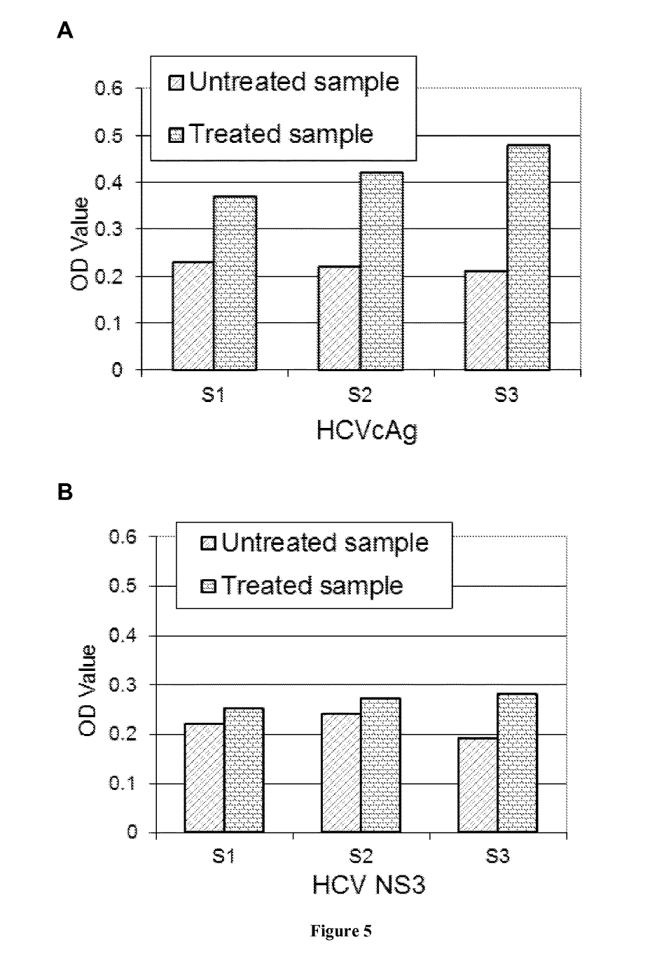

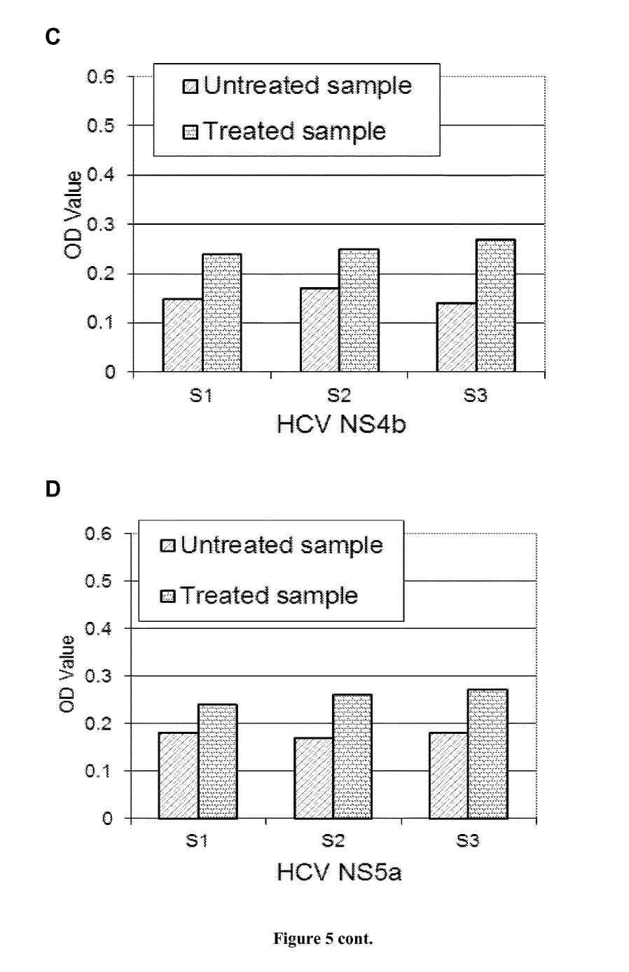

To determine whether the addition of a second HCVcAg detection antibody could further increase assay sensitivity of the combo-HCV-Ags EIA (wherein the plurality of free HCV antigens being detected includes HCVcAg, NS3, NS4b, and NS5a), samples from subjects testing positive for HCV RNA were tested using the EIA protocol of Example 2 with Step 5, and a second anti-HCVcAg detection antibody. As shown in the bar graphs of FIG. 4, the addition of the second anti-HCVcAg detection antibody to the combo-HCV-Ags EIA assay (Example 2) for serum specimens increases the assay sensitivity (2 Core+Combo vs. 1 Core+Combo), and is superior to an EIA assay measuring only the HCVcAg using two different antibodies specific to HCVcAg (2 Core+Combo vs. 2 Core).

Therefore, in some embodiments, more than one antibody, e.g., a second antibody, against the same antigen is used in the combo-HCV-Ags EIA assays, systems, and kits of the present invention. In some embodiments, both the first antibody and the second antibody are monoclonal antibodies or polyclonal antibodies. In some embodiments, the first antibody is a monoclonal antibody and the second antibody is a polyclonal antibody. In some embodiments, the first antibody and the second antibody are capture antibodies, detection antibodies, or both. In some embodiments, the first and second antibodies specifically bind an antigen selected from the group consisting of HCVcAg, E1, E2, NS2, NS3, NS4a, NS4b, NS5a, and NS5b. In some embodiments, the first and second antibodies specifically bind HCVcAg. In some embodiments, a second set of first and second antibodies against a second antigen are employed in the combo-HCV-Ags EIA assays, systems, and kits of the present invention. As used herein, an antibody that specifically binds a given antigen is one that is raised against the given antigen or preferentially binds the given antigen over other antigens.

5.5 Mixing Detection Antibodies with Test Sample

To determine whether all the detection antibodies against the plurality of HCV antigens can be mixed together and incubated with the sample being tested, both serum and urine samples from subjects testing positive for HCV RNA were tested using the EIA protocol of Example 2 (with or without Step 5) and with the following modifications: Instead of coating anti-HCV specific antibodies as described in Step 1, the test wells of the microtiter plate were coated with a sufficient amount, 50-200 μL, e.g., about 100 μL, of capture antibodies, e.g., anti-mouse IgG antibodies, diluted in carbonate/bicarbonate buffer (pH about 7.0-9.5, e.g., about 9.0). The concentration was about 0.5-1.5 μg/mL, e.g., about 1.0 μg/mL. The microtiter plate was incubated at 4° C. for overnight (or 37° C. for about 15-120 minutes, e.g., about 60 minutes). Then, instead of Steps 6-8, the sample to be tested was mixed with the first and second detection antibodies against HCVcAg, NS3, NS4b, and NS5a (e.g., comprising two different HCVcAg mAb, and polyclonal antibodies against NS3, NS4b, and NS5a) before loading into the test well. Briefly, about 25-150 μL, e.g., about 100 μL, of the test sample (plasma or urine) was mixed with all the detection antibodies each at concentration of about 5-20 μg/mL, e.g., about 10 μg/mL, to a total of about 100-300 μL, e.g., about 250 μL final volume. After removing the blocking buffer from the test well, the test mixture (test sample mixed with the detection antibodies) was added, at a volume of about 150-300 μL, e.g., about 250 μL, to the test well having the capture antibodies thereon.

As shown FIG. 10A, mixing all the antibodies together and incubating with the test sample significantly increased assay sensitivity and shortened the test time by about 30 minutes. Therefore, the present invention also provides an EIA assay method, wherein the test sample is mixed with the detection antibodies and then the mixture is added to the assay substrate having the capture antibodies coated or immobilized thereon. In some embodiments, the combo-HCV-Ags EIA assay method of the present invention comprises mixing the test sample and the detection antibodies and then adding the mixture to the assay substrate having the capture antibodies coated or immobilized thereon instead of separately loading the test sample and then the detection antibodies. In some embodiments, the present invention provides kits for performing EIA assays, which comprise a container comprising a plurality of antibodies wherein each antibody in the plurality specifically binds an HCV antigen of a plurality of HCV antigens, a container comprising a detection reagent, and a container wherein the plurality of antibodies, the detection reagent, and the sample to be tested can be mixed, and a substrate having a capture reagent that specifically binds the plurality of HCV antigens coated or immobilized thereon. In some embodiments, the container for mixing is the container having the plurality of antibodies or the container having the detection reagent.

The detection limits of this combo-HCV-Ags EIA assay in which the test sample is mixed with all the detection antibodies prior to being contacted with the assay substrate having capture antibodies (anti-mouse IgG antibodies) coated thereon was determined using serial dilutions of two serum samples having known amounts of HCV RNA. A serum sample that was negative for anti-HCV and HCV RNA by PCR was used as a negative control. The undiluted serum (control) had baseline serum HCV RNA of 1,124-1,140 IU/mL. As shown in FIG. 10B, HCV antigens remained detectable at dilution to serum HCV RNA equivalent to about 140 IU/mL, whether or not the sample was denatured according to Step 5 of Example 2 (the OD values were higher for denatured samples, but the detection limits are comparable for both denatured and non-denatured samples).

Similarly, the detection limits of the combo-HCV-Ags EIA assay in which a urine test sample is mixed with all the detection antibodies prior to being contacted with the assay substrate having capture antibodies (anti-mouse IgG antibodies) coated thereon was determined using serial dilutions of 5 urine samples from subjects having known amounts of serum HCV RNA by RT PCR. A urine sample, from a subject testing negative for anti-HCV and serum HCV RNA by PCR, was used as a negative control. As shown in FIG. 10C, the detection limits were in the range equivalent to serum HCV RNA of about 63-94 IU/mL.

### Example 6—LFT Experiments

Although all the LFT experiments exemplified herein detect only free antigen(s) because the samples are not subjected to denaturing conditions prior to detection, total antigen(s) may be detected by denaturing a test sample, e.g., serum sample, prior to detection.

### 6.1 Detection of Plurality of HCV Antigens

To determine the feasibility of detecting HCVcAg by itself or a plurality of HCV antigens (e.g., HCVcAg, NS3, NS4b, and NS5a) using an LFT assay system, test strips were constructed and tested as set forth in Example 3.

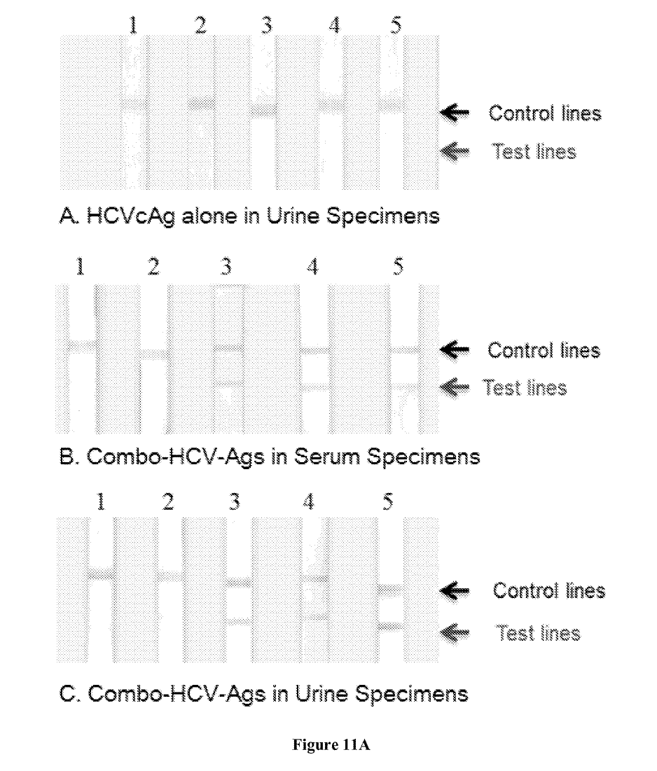

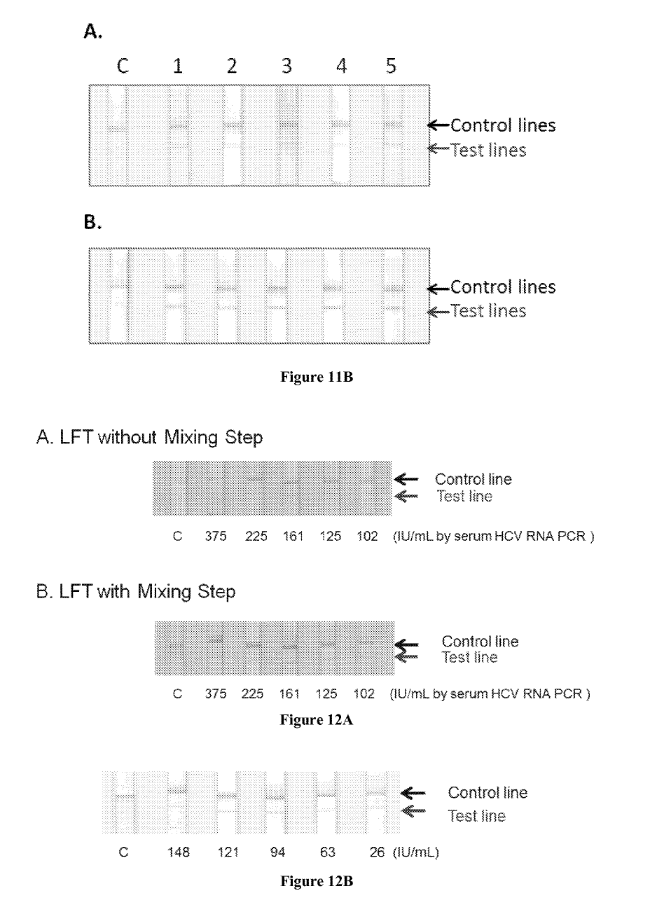

As shown in FIG. 11A, panel A, the LFT assay system employing a single monoclonal antibody against HCVcAg was insufficient to result in a positive signal in urine samples obtained from subjects having high titers of serum HCV RNA, e.g., 14,400,000 IU/mL. However, when using a combo-HCV-Ags LFT assay system according to the present invention—test strips comprising antibodies against the plurality of HCV antigens—positive signals for both serum (FIG. 11A, panel B) and urine (FIG. 11A, panel C) samples from subjects having serum HCV RNA were obtained. Columns 1 and 2 were negative controls.

Therefore, in some embodiments, the present invention provides LFT test strips, which comprise capture and detention antibodies specific against at least two different HCV antigens selected from the group consisting of HCVcAg, E1, E2, NS2, NS3, NS4a, NS4b, NS5a, and NS5b. In some embodiments, the present invention provides LFT test strips, which comprise capture and detention antibodies against HCVcAg and one or more antigens selected from the group consisting of E1, E2, NS2, NS3, NS4a, NS4b, NS5a, and NS5b. In some embodiments, the present invention provides LFT test strips, which comprise capture and detection antibodies against HCVcAg, NS3, NS4b, and NS5a.

### 6.2 Detection of HCVcAg Using Two Different Antibodies

To determine whether the use of an additional antibody against HCVcAg could increase the sensitivity of the combo-HCV-Ags LFT assay of Example 6.2, test strips were constructed and tested as set forth in Example 3, and a second antibody against HCVcAg was added to the mixtures of antibodies against the plurality of HCV antigens. As shown FIG. 11B, the addition of a second antibody against HCVcAg increases the sensitivity of combo-HCV-Ags LFT assays according to the present invention (Panel B with 2 anti-HCVcAg mAbs vs. Panel A with 1 anti-HCVcAg mAb) and the results are HCV genotype independent (Column C, control; strips 1-5 represented HCV GT 1, 2, 3, 4, and 6, respectively).

Therefore, in some embodiments, more than one antibody, e.g., a second antibody, against the same antigen is used in the combo-HCV-Ags LFT assays, systems, and kits of the present invention. In some embodiments, both the first antibody and the second antibody are monoclonal antibodies or polyclonal antibodies. In some embodiments, the first antibody is a monoclonal antibody and the second antibody is a polyclonal antibody. In some embodiments, the first antibody and the second antibody are capture antibodies, detection antibodies, or both. In some embodiments, the first and second antibodies specifically bind an antigen selected from the group consisting of HCVcAg, E1, E2, NS2, NS3, NS4a, NS4b, NS5a, and NS5b. In some embodiments, the first and second antibodies specifically bind HCVcAg. In some embodiments, a second set of first and second antibodies against a second antigen are employed in the combo-HCV-Ags LFT assays, systems, and kits of the present invention.

Therefore, in some embodiments, the present invention provides LFT test strips that comprise more than one antibody, e.g., a second antibody, against the same antigen. In some embodiments, both the first antibody and the second antibody are monoclonal antibodies or polyclonal antibodies. In some embodiments, the first antibody is a monoclonal antibody and the second antibody is a polyclonal antibody. In some embodiments, the first antibody and the second antibody are capture antibodies, detection antibodies, or both. In some embodiments, the first and second antibodies specifically bind an antigen selected from the group consisting of HCVcAg, E1, E2, NS2, NS3, NS4a, NS4b, NS5a, and NS5b. In some embodiments, the first and second antibodies specifically bind HCVcAg. In some embodiments, a second set of first and second antibodies against a second antigen are employed in the combo-HCV-Ags LFT test strips of the present invention.

6.3 Mixing Golden-Conjugated Solution with Test Samples

To determine whether the detection antibodies against the plurality of HCV antigens can be mixed with the sample to be tested rather than being pre-loaded on the test strip without a loss of assay sensitivity and specificity of the combo-HCV-Ags LFT assays according to the present invention, Step 3 of Example 3 was omitted. To perform the assay using the test strips without detection antibodies conjugated with colloid gold on the conjugate pads, test samples were mixed with the colloid gold solution generated in Example 3, Step 1, and then added to the sample pads.

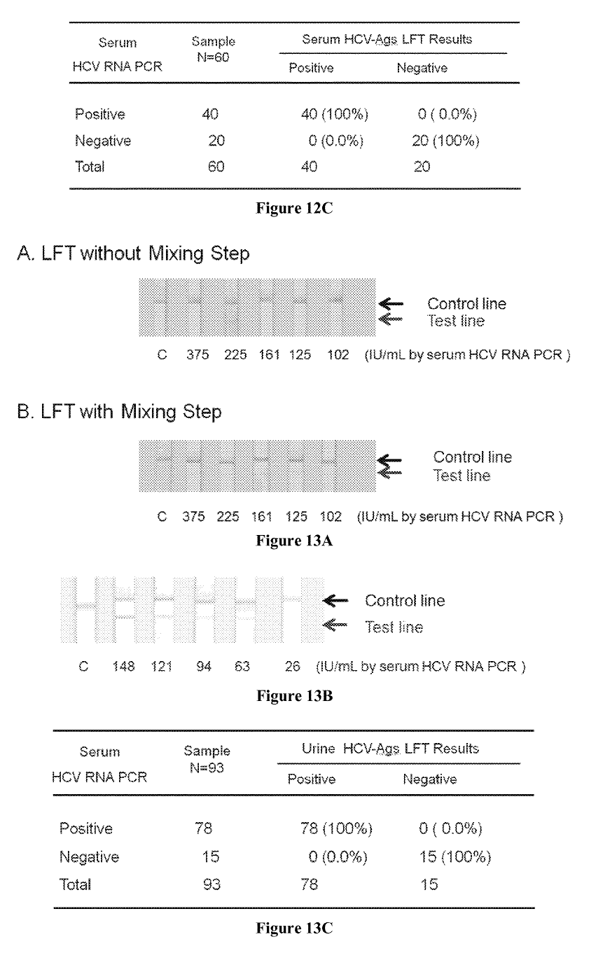

As shown in FIGS. 12A and 12B, mixing the test serum samples with the colloid gold solution (detection antibodies conjugated with colloid gold) before adding to the sample pad significantly improved the sensitivity of the combo-HCV-Ags LFT assays according to the present invention for serum test samples with the detection limit in the range equivalent to serum HCV RNA level of 26-63 IU/mL. The combo-HCV-Ags LFT assays exhibits 100% sensitivity and 100% specificity using serum samples (FIG. 12C).

Similarly, as shown in FIGS. 13A and 13B, mixing the test urine samples with the colloid gold solution (detection antibodies conjugated with colloid gold) before adding to the sample pad significantly improved the sensitivity of the combo-HCV-Ags LFT assays according to the present invention for urine test samples with the detection limit in the range equivalent to serum HCV RNA level of 63-94 IU/mL. The combo-HCV-Ags LFT assays exhibits 100% sensitivity and 100% specificity using urine samples (FIG. 13C).

Therefore, the present invention also provides an LFT assay method, wherein the test sample is mixed with the detection antibodies conjugated with a detectable label, e.g., colloid gold, before adding to the LFT sample pad of the test strip. In some embodiments, the combo-HCV-Ags LFT assay method of the present invention comprises mixing the test sample and the detection antibodies conjugated with a detectable label, e.g., colloid gold, and then adding the mixture to the sample pad of the test strip instead of using a test strip loaded with the detection antibodies conjugated with the detectable label and adding an unmixed test sample to the sample pad. In some embodiments, however, a test strip loaded with the detection antibodies conjugated with a detectable label, e.g., colloid gold, is used to test a test sample having been mixed with the detection antibodies conjugated with the detectable label.

In some embodiments, the present invention provides kits for performing LFT assays that comprise a test strip packaged together with a composition comprising a detectable label, e.g., a colloid gold solution, antibodies, and a container wherein the detectable label can be mixed with the antibodies to result in detection antibodies conjugated with the detectable label, and mixed with the sample to be tested before loading on the test strip. In some embodiments, the kits comprise a test strip packaged together with detection antibodies conjugated to a detectable label, e.g., colloid gold, and a container wherein the sample to be tested and the detection antibodies may be mixed. The test strips provided in the kits may or may not be pre-loaded with detection antibodies conjugated with a detectable label, e.g., colloid gold.

In some embodiments, the kits are for the detection of active HCV infection and therefore the detection antibodies comprise a mixture of antibodies, which specifically bind at least two HCV antigens selected from the group consisting of HCVcAg, E1, E1, NS2, NS3, NS4a, NS4b, NS5a, and NS5b, e.g., said mixture comprises a first antibody specific against a first HCV antigen, a second antibody specific against a second HCV antigen, etc.

### Example 7—HBV Assays Using Two Different Antibodies Against HBsAg

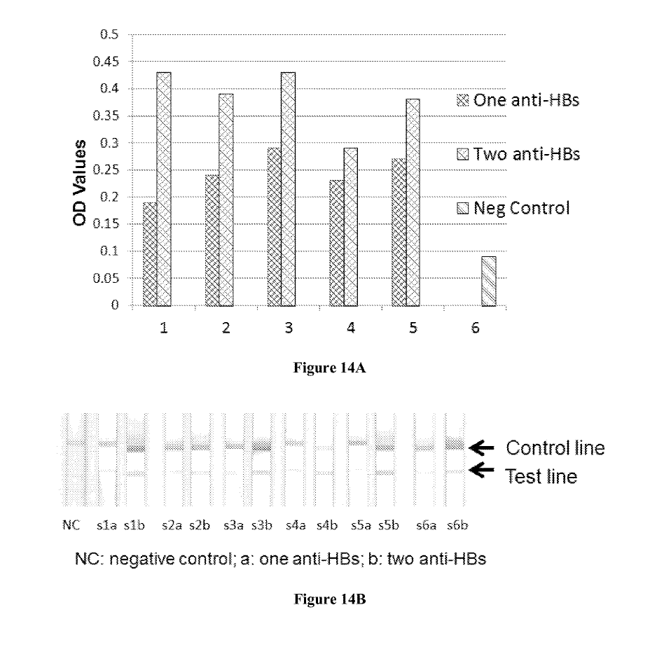

The presence of Hepatitis B Virus surface antigen (HBsAg) in urine samples from subjects positive for serum HBsAg. Specifically, the EIA protocol of Example 5.5 (mixing the sample with the detection antibodies prior to contact with the assay substrate having the capture antibodies thereon) without Step 5 and using one or two antibodies against HBsAg. The antibodies against HBsAg are set forth in Table A. Other HBV antigens were not detected. As shown in FIG. 14A, the use of two different antibodies against HBsAg increased assay sensitivity by about 0.26 to 1.6 times.

The presence of HBsAg in urine samples from subjects positive for serum HBsAg. Specifically, the LFT protocol of Example 6.3 (mixing the sample with the detection antibodies prior to contact with the test strip) and using one or two antibodies against HBsAg. The HBsAg antibodies are set forth in Table A. Other HBV antigens were not detected. As shown in FIG. 14B, LFT assays using two different antibodies against HBsAg (see test strips labeled with “b”) resulted in significantly superior results compared to LFT assays using only one antibody against HBsAg (see test strips labeled with “a”).

Therefore, in some embodiments, the present invention provides immune-based assays, systems, and kits for detecting HBsAg in samples, which comprise the use of at least two different antibodies, e.g., a first antibody against HBsAg and a second antibody against HBsAg. In some embodiments, both the first antibody and the second antibody are monoclonal antibodies or polyclonal antibodies. In some embodiments, the first antibody is a monoclonal antibody and the second antibody is a polyclonal antibody. In some embodiments, the first antibody and the second antibody are capture antibodies, detection antibodies, or both. In some embodiments, the first and second antibodies are mixed with the test sample prior to contact with the assay substrate having capture antibodies thereon. In some embodiments, the sample is denatured prior to detection. In some embodiments, the sample is a urine sample. In some embodiments, the sample is a whole blood, serum, or plasma sample. In some embodiments, the assay is an EIA assay. In some embodiments, the assay is an LFT assay.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. An assay for identifying a sample as containing hepatitis virus antigens, which comprises
contacting the sample with a plurality of antibodies wherein each antibody in the plurality specifically binds a hepatitis virus antigen of a plurality of hepatitis virus antigens,
detecting the presence or absence of any hepatitis virus antigens bound to the antibodies of the plurality of antibodies,
optionally measuring any hepatitis virus antigens bound to the antibodies of the plurality of antibodies, and
identifying the sample as containing hepatitis virus antigens where hepatitis virus antigens bound to the antibodies of the plurality of antibodies are detected as being present, and identifying the sample as not containing hepatitis virus antigens where hepatitis virus antigens bound to the antibodies of the plurality of antibodies are absent,
wherein the hepatitis virus antigens are hepatitis C virus (HCV) antigens, hepatitis B virus (HBV) antigens, or both.

2. The assay of claim 1, wherein the sample is subjected to a condition that disassociates immune complexes prior to the detecting step.

3. The assay of claim 1, wherein the sample is not subjected to a condition that disassociates immune complexes prior to the detecting step.

4. The assay of claim 1, wherein the sample is urine.

5. The assay of claim 1, wherein the sample is whole blood, serum, or plasma.

6. The assay of claim 1, wherein the HCV antigens are selected from the group consisting of HCVcAg, E1, E2, NS2, NS3, NS4a, NS4b, NS5a, and NS5b.

7. The assay of claim 1, wherein the HCV antigens are selected from the group consisting of HCVcAg, E1, E2, NS2, NS3, NS4a, NS4b, NS5a, and NS5b, and at least one of the HCV antigens is HCVcAg.

8. The assay of claim 1, wherein the HCV antigens comprise, consist essentially of, or consist of HCVcAg, NS3, NS4b, and NS5a.

9. The assay of claim 1, wherein the plurality of antibodies comprises a first antibody and a second antibody, said first and second antibodies specifically bind the same hepatitis virus antigen.

10. The assay of claim 1, and further comprises mixing the sample with the plurality of antibodies to form a mixture and then contacting the mixture with a substrate having a capture reagent that specifically binds the plurality of antibodies, which may or may not be bound to the hepatitis virus antigens before the detecting step.

11. The assay of claim 1, wherein the detecting step comprises attaching a detectable label to each antibody of the plurality of antibodies.

12. A method of diagnosing a subject as having an active hepatitis virus infection, which comprises
diagnosing the subject as having an active hepatitis C virus (HCV) infection where a sample from the subject has been identified as containing HCV antigens according to the assay method of claim 3,
diagnosing the subject as having an active hepatitis B virus (HBV) infection where the sample from the subject has been identified as containing HBV antigens according to the assay method of claim 3, or
diagnosing the subject as not having an active hepatitis virus infection where the sample from the subject has been identified as not containing hepatitis virus antigens according to the assay method of claim 3.

13. A method of identifying a subject, from a plurality of subjects, as having or not having an active hepatitis virus infection, which comprises
testing samples from the plurality of subjects according to the assay method of claim 3, and
identifying the subject as having an active hepatitis C virus (HCV) infection where a sample from the subject has been identified as containing HCV antigens,
identifying the subject as having an active hepatitis B virus (HBV) infection where the sample from the subject has been identified as containing HBV antigens, or
identifying the subject as not having an active hepatitis virus infection where the sample from the subject has been identified as not containing hepatitis virus antigens.

14. A method for diagnosing a subject as having an active hepatitis virus infection or having had a past and cleared hepatitis virus infection, which comprises
obtaining a first and a second sample from the subject, wherein the first sample may or may not be capable of having immune complexes and the second sample is capable of having immune complexes,
performing the assay of claim 1 on the first sample, which is either not capable of having immune complexes and/or has not been subjected to conditions that disassociate immune complexes,
performing the assay of claim 1 on the second sample, which has been subjected to conditions that disassociate immune complexes, with the plurality of antibodies, and
diagnosing the subject as having an active hepatitis virus infection where hepatitis virus antigens bound to the antibodies of the plurality of antibodies are detected as being present in the first sample, and diagnosing the subject as having a past and cleared hepatitis virus infection where hepatitis virus antigens bound to the antibodies of the plurality of antibodies are detected in the second sample and no hepatitis virus antigens are detected in the first sample,
wherein the hepatitis virus is hepatitis C virus (HCV) and the hepatitis virus antigens are HCV antigens or the hepatitis virus is hepatitis B virus (HBV) and the hepatitis virus antigens are HBV antigens.

15. A method of monitoring a subject who had, has, or may have an active hepatitis virus infection, which comprises
obtaining a first and a second sample from the subject, wherein at least the second sample is capable of having immune complexes, at a first point in time,
performing the assay of claim 1 on the first sample, which is either not capable of having immune complexes and/or has not been subjected to conditions that disassociate immune complexes,
performing the assay of claim 1 on the second sample, which has been subjected to conditions that disassociate immune complexes,
obtaining a third and a fourth sample from the subject, wherein at least the fourth sample is capable of having immune complexes, at a second point in time,
performing the assay of claim 1 on the third sample, which is either not capable of having immune complexes and/or has not been subjected to conditions that disassociate immune complexes,
performing the assay of claim 1 on the fourth sample, which has been subjected to conditions that disassociate immune complexes, and
calculating the differences in hepatitis virus antigens bound to the antibodies of the plurality of antibodies between the first, second, third, and fourth samples,
wherein the hepatitis virus is hepatitis C virus (HCV) and the hepatitis virus antigens are HCV antigens or the hepatitis virus is hepatitis B virus (HBV) and the hepatitis virus antigens are HBV antigens.

16. (canceled)

17. (canceled)

18. (canceled)

19. A lateral flow test substrate having a sample loading area, a test area, and a control area, wherein a capture reagent is immobilized in the test area, said capture reagent is a plurality of antibodies wherein each antibody in the plurality specifically binds a hepatitis virus antigen of a plurality of hepatitis virus antigens,
wherein the hepatitis virus antigen is hepatitis C virus (HCV) antigen, hepatitis B virus (HBV), or both.

20. A kit comprising
the lateral flow test substrate of claim 19, and
a detection reagent.

21. An immunoassay for an analyte in a test sample, which comprises
mixing the test sample with one or more detection antibodies which specifically bind the analyte and then contacting the mixture with an assay substrate having capture antibodies which specifically bind the analyte coated or immobilized thereon the surface of the assay substrate.

22. (canceled)

23. The assay of claim 21, wherein the analyte is HBsAg and a first antibody and a second antibody, said first and second antibodies specifically bind HBsAg, are used.

24. A method of diagnosing a subject as having an active hepatitis virus infection, which comprises
diagnosing the subject as having an active hepatitis C virus (HCV) infection where a sample from the subject has been identified as containing HCV antigens according to the assay method of claim 4,
diagnosing the subject as having an active hepatitis B virus (HBV) infection where the sample from the subject has been identified as containing HBV antigens according to the assay method of claim 4, or
diagnosing the subject as not having an active hepatitis virus infection where the sample from the subject has been identified as not containing hepatitis virus antigens according to the assay method of claim 4.
